(19) United States
(12) Patent Application Publication
(10) Pub. No.: US 2018/0271808 A1
(43) Pub. Date: Sep. 27, 2018

(54) METHODS OF REDUCING CHEMORESISTANCE AND TREATING CANCER

(71) Applicant: Mitchell Woods Pharmaceuticals, Inc., Shelton, CT (US)

(72) Inventor: Irving W. Wainer, Washington, DC (US)

(21) Appl. No.: 15/765,082

(22) PCT Filed: Sep. 30, 2016
(86) PCT No.: PCT/US16/54819

§ 371 Date: Mar. 30, 2018

Related U.S. Application Data
(60) Provisional application No. 62/235,853, filed on Oct. 1, 2015.

(51) Int. Cl. A61K 31/137; A61K 45/06; G01N 33/574; A61P 35/00

(52) U.S. Cl. CPC A61K 31/137; A61P 35/00; G01N 33/574; A61K 45/06

(57) ABSTRACT

Methods of treating cancer include administering a therapeutically effective amount of a pharmaceutical composition containing a pharmaceutically acceptable carrier and a compound that is a GPR55 antagonist to a subject having a tumor including cancer cells that have developed a resistance to one or more chemotherapeutic agents. In embodiments, the compound is a fenoterol analogue, such as for example, MNF.

### CROSS REFERENCE TO RELATED APPLICATION

This application claims priority to and the benefit of U.S. Provisional Application Ser. No. 62/235,853 filed on Oct. 1, 2015, the entire contents of which are incorporated herein by reference.

### TECHNICAL FIELD

The present disclosure relates to methods of treating cancer by inhibiting GPR55-mediated activation of select cancer biomarkers within tumors by administration of at least one agent, such as for example a fenoterol analogue. By inhibiting activation of the biomarkers, the fenoterol analogue suppresses multidrug resistance to chemotherapeutic agents in tumors, including pancreatic cancer tumors.

### BACKGROUND

Cancer is the second leading cause of human death next to coronary disease in the United States. Worldwide, millions of people die from cancer every year. In the United States alone, as reported by the American Cancer Society, cancer causes the death of well over a half-million people annually, with over 1.2 million new cases diagnosed per year. While deaths from heart disease have been declining significantly, those resulting from cancer generally are on the rise. Cancer is soon predicted to become the leading cause of death.

### SUMMARY

This disclosure concerns the discovery that inhibiting GPR55-mediated activation of select cancer biomarkers in cancer cells reduces the expression of proteins key to the resistance of tumors to treatment by anticancer drugs. In embodiments, fenoterol analogues are used to treat cancer by inhibiting GPR55-mediated activation of select cancer biomarkers in tumors. The exemplary methods described herein can be used, for example, to treat pancreatic cancer, breast cancer, or other cancers.

In embodiments, the method includes administering a therapeutically effective amount of a pharmaceutical composition containing a pharmaceutically acceptable carrier and a compound that is a GPR55 antagonist to a subject having tumors including cancer cells that have developed a resistance to one or more chemotherapeutic agents, thereby suppressing multidrug resistance of tumor cells (such as human pancreatic cancer cells) to chemotherapeutic agents.

In embodiments, the pharmaceutical composition is administered to a subject having either pancreatic cancer tumors or breast cancer tumors that include cells that have developed a resistance to one or more chemotherapeutic agents. In embodiments, the pharmaceutical composition is administered to a subject having tumors including cancer cells that have developed a resistance to at least one of the commonly used chemotherapeutic agents, such as doxorubicin or gemcitabine.

In embodiments, the pharmaceutical composition includes a fenoterol analogue. In embodiment, the pharmaceutical composition includes one or more compounds selected from the group consisting of (R,R′)-4′-methoxy-1-naphthylfenoterol (“MNF”), (R,S′)-4′-methoxy-1-naphthylfenoterol, (R,R′)-ethylMNF, (R,R′)-napthylfenoterol, (R,S′)-napthylfenoterol, (R,R′)-ethyl-naphthylfenoterol, (R,R′)-4′-amino-1-naphthylfenoterol, (R,R′)-4′-hydroxy-1-naphthylfenoterol, (R,R′)-4-methoxy-ethylfenoterol, (R,R′)-methoxyfenoterol, (R,R′)-ethylfenoterol, (R,R′)-fenoterol, and their respective stereoisomers.

In embodiments, the fenoterol analogue is (R,R′)-4′-methoxy-1-naphthyl-fenoterol (MNF), a compound having the formula:

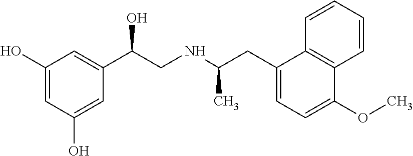 [SMILES: COc1ccc(C[C@@H](C)NC[C@H](O)c2cc(O)cc(O)c2)c2ccccc12]

In embodiments, the presently described methods include administering a therapeutically effective amount of a pharmaceutical composition containing a fenoterol analogue and a pharmaceutically acceptable carrier to a cancer patient to inhibit GPR55-mediated activation of select cancer biomarkers in a tumor. In embodiments, the cancer patient is known to have pancreatic cancer. In embodiments, the cancer patient is known to have a tumor including cancer cells that have developed resistance to a chemotherapeutic agent. In embodiments, the method includes administering one or more therapeutic agents in addition to a fenoterol analogue. The methods can include administration of the one or more therapeutic agents separately, sequentially or concurrently, for example in a combined composition with a fenoterol analogue. In embodiments, the one or more therapeutic agents administered in addition to a fenoterol analogue is a chemotherapeutic agent.

In embodiments, the pharmaceutical composition administered attenuates both the constitutive and ligand-inducible activity of the MEK/ERK and PI3K/AKT pathways. In embodiments, the pharmaceutical composition administered downregulates multidrug resistance (“MDR”) protein expression. In embodiments, the pharmaceutical composition administered decreases HIF-1α and β-catenin levels in the cancer cells. In embodiments, the pharmaceutical composition administered results in increased nuclear accumulation of one or more chemotherapeutic agents in the cancer cells. In embodiments, the pharmaceutical composition administered results in increased cytotoxicity of one or more chemotherapeutic agents with respect to the cancer cells.

In another aspect, methods in accordance with the present disclosure include identifying a subject having a tumor including cancer cells that have developed a resistance to one or more chemotherapeutic agents, and administering a therapeutically effective amount of a pharmaceutical composition containing a pharmaceutically acceptable carrier and a compound that is a GPR55 antagonist to the subject. In embodiments, the subject identified has either a pancreatic tumor or a breast cancer tumor including cancer cells that have developed a resistance to one or more chemotherapeutic agents. In embodiments, the pharmaceutical composition is administered to a subject having a tumor including cancer cells that have developed a resistance to at least one of doxorubicin or gemcitabine. In embodiments, the method includes administering a chemotherapeutic agent before, after or during administration of the pharmaceutical composition. In embodiments, the anticancer drug selected from the group consisting of doxorubicin, daunorubicin, mitoxantrone, paclitaxel, docetaxel, vincristine, vinblastine, cisplatin, methotrexate, gemcitabine, 5-fluorouracil, bortezomib, carfilzomib, erlotinib, ceritinib, sunitib, imatinib, irinotecan, topotecan, palbociclib, and vemurafenib. In embodiments, the pharmaceutical composition is administered before, after, or during administration of the chemotherapeutic agent to which the cancer cells of the tumor have developed a resistance. In embodiments, the pharmaceutical composition administered contains one or more compounds selected from the group consisting of (R,R′)-4′-methoxy-1-naphthylfenoterol (“MNF”), (R,S′)-4′-methoxy-1-naphthylfenoterol, (R,R′)-ethylMNF, (R,R′)-napthylfenoterol, (R,S′)-napthylfenoterol, (R,R′)-ethyl-naphthylfenoterol, (R,R′)-4′-amino-1-naphthylfenoterol, (R,R′)-4′-hydroxy-1-naphthylfenoterol, (R,R′)-4′-methoxy-ethylfenoterol, (R,R′)-4′-methoxyfenoterol, (R,R′)-ethylfenoterol, (R,R′)-fenoterol and their respective stereoisomers. In embodiments, the pharmaceutical composition administered contains a compound of the formula:

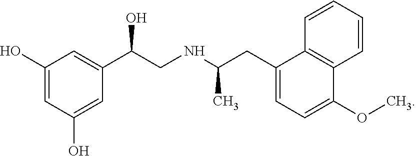 [SMILES: COc1ccc(C[C@@H](C)NC[C@H](O)c2cc(O)cc(O)c2)c2ccccc12]

In embodiments, administering the pharmaceutical composition results in increased nuclear accumulation of one or more chemotherapeutic agents in the cancer cells of a tumor. In embodiments, administering the pharmaceutical composition results in increased cytotoxicity of one or more chemotherapeutic agents with respect to the cancer cells of a tumor.

According to exemplary methods described herein, administration of fenoterol analogues suppresses multidrug resistance of tumor cells (such as human pancreatic cancer cells) to chemotherapeutic agents and, therefore, allows chemotherapy to kill a higher proportion of drug-sensitive cancer cells, reducing the number of cancer cells available for growth and spread of the cancer.

The foregoing simplified summary is presented in order to provide a basic understanding of some aspects of the claimed subject matter. The foregoing summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented hereinbelow. Further scope of applicability of the present disclosure will become apparent from the detailed description given hereinafter. However, it should be understood that the detailed description and specific examples, while indicating specific embodiments of the present disclosure, are given by way of illustration only, since various changes and modifications within the spirit and scope of the present disclosure will become apparent to those skilled in the art from this detailed description.

### BRIEF DESCRIPTION OF THE DRAWINGS

FIG. 1 shows that MNF treatment reduces human derived pancreatic cancer cell line (PANC-1) cell proliferation. Thymidine incorporation was assessed into PANC-1 cells treated with increasing concentrations of MNF (0-10 μM) for 24 hours. Data in FIG. 1 are expressed as mean±SD from 3 independent experiments.

FIGS. 2A-D show that MNF dose-dependently reduces expression of cancer biomarkers in PANC-1 tumor cells. Specifically, FIG. 2A presents representative immunoblot showing protein levels of EGFR, β-catenin, PKM2, cyclin D1, and β-actin in PANC-1 cells at 24 hours after MNF treatment. In FIG. 2B, protein bands were quantified by densitometry and the data normalized to β-actin levels and plotted relative to cells treated with 0.01% DMSO as control. FIG. 2C presents a representative experiment showing protein levels of Pgp, BCRP, MRP1, MRP5, and β-actin in PANC-1 cells at 24 hours after MNF treatment. FIG. 2D presents densitometric analysis of the protein bands performed as in FIG. 2B. Data in FIGS. 2A-D are expressed as mean±SD from 3 independent experiments. *, **, *** P<0.05, <0.01, and <0.001 vs. control.

FIGS. 3A-F demonstrate that GPR55 promotes tumorigenesis by upregulating expression of cancer biomarkers. Specifically, FIG. 3A presents representative immunoblot depicting protein levels of EGFR, BCRP, PKM2, and β-actin in PANC-1 cell lysates at 24 hours after cell pretreatment with MNF (1 μM) or the GPR55 antagonist CID 16020046 (CID, 1 μM) for 30 minutes followed by treatment without or with the GPR55 agonist AM251 (1 μM). In FIG. 3B, protein bands were quantified by densitometry and the data normalized to β-actin levels and plotted relative to cells treated with 0.01% DMSO as control. FIG. 3C presents representative immunoblot depicting phosphoactive (pSer) and total forms of AKT in PANC-1 cells after pretreatment with MNF or CID for 10 minutes followed by a 20 minute incubation with and without AM251. FIG. 3D presents densitometric analysis of the protein bands performed and the ratio pSer-AKT/total AKT calculated and plotted relative to cells treated with 0.01% DMSO as control. FIG. 3D presents representative immunoblot depicting protein levels of phospho-active forms of PKM2 (pPKM2) and β-catenin (p β-catenin), and those of HIF-1α and lamin A/C in PANC-1 nuclear extracts after cell pretreatment with MNF or CID for 10 minutes, followed by a 20 minute treatment with and without AM251. FIG. 3F presents densitometric analysis performed as in FIG. 3B and the data normalized to lamin A/C levels. Data in FIGS. 3A-F are expressed as mean±SD from 3 independent experiments. *, **, ***P<0.05, <0.01, <0.001 vs. control;

FIG. 4 shows that GPR55 targets phospho-active PKM2 to the nucleus in PANC-1 cells. Specifically, FIG. 4 presents densitometric analysis indicating the nuclear levels of p-PKM2 normalized to control samples. Data in FIG. 4 are expressed as mean±SD from 3 independent experiments. *** P<0.001 vs. control.

FIGS. 5A-D demonstrate that MNF increases the cytotoxicity of chemotherapeutic drugs by suppressing expression of multidrug resistance proteins. Specifically, FIG. 5A shows thymidine incorporation into PANC-1 cells treated with MNF (1 μM) for 24 hours and followed with 0-10 μM of doxorubicin for 24 hours. FIG. 5B shows thymidine incorporation into PANC-1 cells as in FIG. 5A, but with 0-10 μM of gemcitabine for 24 hours. Values were normalized to PANC-1 cells treated with 0.01% DMSO as control. Data are expressed as mean±SD from 3 independent experiments. FIG. 5C presents fluorescence imaging of intracellular doxorubicin in PANC-1 cells pretreated with MNF (1 μM) for 24 hours and followed with 0.125 μM of doxorubicin treatment for 2 hours. The upper panel of FIG. 5D presents representative immunoblot depicting protein levels of Pgp, BCRP, and lamin A/C in PANC-1 nuclear extracts after cell treatment with 0.5 and 1 μM of MNF for 24 hours. In the lower panel of FIG. 5D, protein bands were quantified by densitometry and the data normalized to lamin A/C levels and plotted relative to cells treated with 0.01% DMSO as control.

FIGS. 6A-E demonstrate that MNF lowers mitogenesis and reduces chemoresistance in cancer cell lines. Specifically, in FIG. 6A, thymidine incorporation was carried out in human MCF-7 and MDA-MB-231 breast cancer cell lines after a 24 hour incubation with increasing concentrations of MNF. Bars represent the mean±SD (n=3 triplicate dishes) of a representative experiment. FIG. 6B shows fluorescence imaging of intracellular doxorubicin in MDA-MB-231 cells pretreated with vehicle or MNF (1 μM) for 24 hours, followed with 0.125 μM of doxorubicin treatment for 2 hours. FIG. 6C shows the quantitative determination of fluorescence intensity from three independent experiments, *** P<0.001. FIG. 6D shows thymidine incorporation into MDA-MB-231 cells treated without or with MNF (1 μM) for 24 hours and followed with 0-10 μM of doxorubicin for 24 hours. Data points represent the mean±SD (n=3 triplicate dishes) of a representative experiment. FIG. 6E shows thymidine incorporation into human U87MG glioma cells treated without or with MNF (0.01 μM) for 24 hours, followed with the indicated concentrations of doxorubicin for 24 hours. Data points represent the mean±SD (n=3 triplicate dishes) of a representative experiment.

FIG. 7 is a diagram depicting the anti-tumorigenic function of GPR55 antagonists and consequent reduction in multidrug resistance.

### DETAILED DESCRIPTION

### Introduction

While there have been significant advances in the treatment of cancer, a number of tumors are resistant to frontline chemotherapeutic agents and patients who receive chemotherapy often relapse with the development of multidrug resistance (MDR). MDR is a complex interrelated phenomenon, which is dependent, in part, upon increased expression and function of ABC transporters such as P-glycoprotein (Pgp, MDR1/ABCB1), multidrug resistance protein 1-7 (MRP1-7, ABCC1-7) and breast cancer resistance protein (BCRP, ABCG2). For example, pancreatic adenocarcinoma (PancCa) is the fourth leading cause of cancer death in the U.S. and has one of the worst clinical prognosis as only 6% of PancCa patients will survive beyond 5 years post diagnosis. MDR contributes to the poor response to frontline treatment of PancCa, as enhanced expression of MRP-1, MRP-3 and MRP-5 has been associated with resistance to gemcitabine and 5-fluorouracil. In the treatment of breast cancer, over 80% of the breast cancer patients who receive chemotherapy will relapse with the eventual development of MDR disease primarily associated with overexpression of Pgp, MRP1 and BCRP.

The expression of the MDR phenotype in tumors has been linked to activation of the epidermal growth factor receptor (EGFR) and the subsequent downstream increase in ERK1/2 phosphorylation (p-ERK), which contributes to increased MDR-related protein expression. Cellular proliferation and MDR have also been associated with increased expression of β-catenin and hypoxia-inducible factor 1α (HIF-1α). Another key player implicated in the upregulation of genes linked to tumor proliferation is pyruvate kinase M2 (PKM2), whose phosphorylation by ERK2 enables its nuclear translocation and association with β-catenin to enhance transactivation of target genes, including cyclin D1 (CCND1). Activated Wnt/β-catenin pathway activity has been found to enhance Pgp expression in chronic myeloidleukemia and cholangiocarcinoma, while the knockdown of β-catenin decreases BCRP expression and augments the antiproliferative effects of 5-fluorouracil in MDA-MB-468 breast cancer cells and down regulates Pgp expression in glioblastoma stem cells. In response to ERK-mediated phosphorylation, PKM2 binds with nuclear HIF-1α to upregulate HIF-1α-dependent transcription. Notably, HIF-1α transactivates Pgp gene expression in breast, colon, and stomach tumors and raises MRP-2 levels in breast cancer cell lines. However, the contribution of PKM2 to the expression and function of MDR proteins through its association with β-catenin and HIF-la pathways remains unclear.

The relationship between increased activity of the MEK/ERK and PI3K-AKT pathways with oncogenesis and elevated MDR protein expression makes these pathways important targets for drug development. One potential approach is through the inhibition of GPR55, a G protein-coupled receptor whose activation is linked to tumorigenesis via increased activity of the MEK/ERK and PI3K-AKT pathways. Elevated expression of GPR55 mRNA has been linked to aggressiveness in human PancCa and glioblastoma tumors, and GPR55 siRNA knockdown reduced growth of a T98G glioblastoma tumor maintained as a subcutaneous tumor in mice. GPR55 expression was detected in MDA-MB-231 breast cancer cell lines and incubation with the endogenous GPR55 agonist 1-α-lysophosphatidylinositol (LPI) increased cellular migration, orientation and polarization. In prostate and ovarian tumor cells, LPI activation of GPR55 increased p-ERK and p-AKT concentrations, which was blocked by pre-incubation with the GPR55 antagonist cannabidiol. In addition, siRNA-mediated GPR55 knockdown and treatment with the GPR55 antagonist CID 16020046 (CID) effectively blocked the LPI-mediated ovarian cancer-induced angiogenesis in an in vivo chicken chorioallantoic assay. It has recently been reported that the human-derived pancreatic cancer cell line “PANC-1” and the human-derived hepatocellular HepG2 cell line express GPR55 mRNA and protein. In PANC-1 and HepG2 cells, knockdown of GPR55 with specific siRNAs abrogates cellular uptake of Tocrifluor 1117, a selective GPR55 ligand. It has also been demonstrated that treatment of these cell lines with (R,R′)-4′-methoxy-1-naphthylfenoterol (MNF) inhibits cellular uptake of Tocrifluor 1117 in a concentration-dependent manner and has a negative impact on GPR55 signaling, as evidenced by the significant impairment in ligand-inducible changes in p-ERK levels, cell morphology, and migration using scratch wound healing assay. In addition, incubation of HepG2 cells with MNF decreased signaling through the MEK/ERK and PI3K/AKT pathways to produce cell cycle arrest and apoptosis.

In view of the ability of MNF to block the inducible expression of EGFR protein by a GPR55 ligand and significantly lower GPR55-mediated activation of the EGFR-MEK/ERK cascade in PANC-1 cells, the present inventors have undertaken an expanded investigation of the effect of MNF on cellular signaling and expression of key tumor biomarkers such as PKM2 and MDR expression and function. It has now been discovered that the pharmacological inhibition of GPR55 using methods in accordance with the present disclosure provides attenuation in PKM2 nuclear accumulation and MDR protein expression in PANC-1 cells. The reduction in both the expression and function of MDR proteins results in increased doxorubicin and gemcitabine cytotoxicity. Incubation with MNF also increased the cytotoxicity of doxorubicinin MDA-MB-231 and U87MG cancer cell lines. Accordingly, in addition to having anti-mitogenic properties, the use of pharmacological inhibitors of GPR55 in accordance with the present disclosure is useful in the treatment of a tumor exhibiting MDR or a tumor that has acquired MDR phenotype.

In accordance with exemplary embodiments of the present disclosure, a GPR55 antagonist, such as a fenoterol analogue, is used to treat a disease state by attenuating both the constitutive and ligand-inducible activity of the MEK/ERK and PI3K/AKT pathways. In embodiments, administration of a GPR55 antagonist, such as a fenoterol analogue, is used to reduce phosphorylation and nuclear translocation of PKM2 and produce a concomitant decrease in HIF-1α and β-catenin levels. In embodiments, administration of a GPR55 antagonist, such as a fenoterol analogue, is used to downregulate MDR protein expression and disrupt feed-forward loops responsible for chemoresistance and survival in tumor cells

(R,R)-4′-methoxy-1-napthylfenoterol (“MNF”) is a potent competitive inhibitor of the G-protein coupled receptor GPR55 and the anti-proliferative effects are associated with down-stream events including attenuation of epidermal growth factor receptor (EGFR) expression and reduced phosphorylation of extracellular signal-related kinases (ERK1/2). It has now been found that MNF also reduces decreased EGFR, PKM2, and β-catenin protein levels and is accompanied by significant reduction in nuclear accumulation of HIF-1α and phospho-active forms of PKM2 and β-catenin in cancer cells, which in turn reduces expression of proteins key to the survival of cancer cells and their resistance to treatment by anticancer drugs (known as multidrug resistance).

Specifically it has been found that fenoterol analogues, such as (R,R)-4′-methoxy-1-napthylfenoterol (MNF), reduce the resistance of pancreatic cancer cells to treatment by anticancer drugs. In particular, a series of studies were performed to characterize fenoterol analogues and determine their possible therapeutic activities. MNF has now been found to exert antitumor effects by inhibiting GPR55-mediated activation of select cancer biomarkers and suppressing multidrug resistance to chemotherapeutic agents in pancreatic cancer cell lines, including PANC-1.

In accordance with other exemplary embodiments of the present disclosure, a fenoterol analogue is used to treat a disease state by inhibiting GPR55-mediated activation of select cancer biomarkers in cells. In embodiments, tumors are treated by inhibiting GPR55-mediated activation of select cancer biomarkers in cancer cells.

For example, fenoterol analogues, such as (R,R)-4′-methoxy-1-napthylfenoterol (MNF), suppress multidrug resistance to chemotherapeutic agents in cancer cells, such as pancreatic cancer cells. PANC1 cells incubated with MNF exhibited significantly decreased EGFR, PKM2, and β-catenin protein levels that was accompanied by significant reduction in nuclear accumulation of HIF-1α and phospho-active forms of PKM2 and B-catenin. Thus, MNF, a compound that decreases EGFR, PKM2, and β-catenin expression and activity, suppresses multidrug resistance to chemotherapeutic agents in cancer cell lines.

The methods, compounds, and compositions described herein can be used to treat pancreatic as well as other forms of cancer. Based upon these findings, methods of treating disorders and diseases using combinations of MNF and other chemotherapeutic agents are disclosed.

### Abbreviations and Terms

Abbreviations:
AM251: 1-(2,4-dichlorophenyl)-5-(4-iodophenyl)-4-methyl-N-(1-piperidyl)pyrazole-3-carboxamide
AM630: 1-[2-(morpholin-4-yl)ethyl]-2-methyl-3-(4-methoxybenzoyl)-6-iodoindole
AR: adrenergic receptor
2-AR: 2-adrenergic receptor
CB: cannabinoid
EGFR: epidermal growth factor receptor
ERK: extracellular regulated kinase
GPR55: G protein-coupled receptor 55
GPCR: G protein-coupled receptor
HPLC: high performance liquid chromatography
ICI 118,551: 3-(isopropylamino)-1-[(7-methyl-4-indanyl)oxy]butan-2-ol
ICYP: [¹²⁵I]cyanopindolol
IP: intraperitoneal
IV: intravenous
MNF: (R,R′)-4-methoxy-1-naphthylfenoterol
NF: naphthylfenoterol
UV: ultraviolet

Terms:

Unless otherwise explained, all technical and scientific terms used herein have the same meaning as commonly understood by one of ordinary skill in the art to which the disclosed subject matter belongs. Definitions of common terms in chemistry may be found in The McGraw-Hill Dictionary of Chemical Terms, 1985, and The Condensed Chemical Dictionary, 1981.

Except as otherwise noted, any quantitative values are approximate whether the word “about” or “approximately” or the like are stated or not. The materials, methods, and examples described herein are illustrative only and not intended to be limiting. Any molecular weight or molecular mass values are approximate and are provided only for description. Except as otherwise noted, the methods and techniques of the present invention are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley—Interscience, 2001; or Vogel, A Textbook of Practical Organic Chemistry, Including Qualitative Organic Analysis, Fourth Edition, New York: Longman, 1978.

In order to facilitate review of the various embodiments disclosed herein, the following explanations of specific terms are provided:

Acyl: A group of the formula RC(O)— wherein R is an organic group.

Acyloxy: A group having the structure —OC(O)R, where R may be an optionally substituted alkyl or optionally substituted aryl. “Lower acyloxy” groups are those where R contains from 1 to 10 (such as from 1 to 6) carbon atoms.

Administration: To provide or give a subject a composition, such as a pharmaceutical composition including one or more fenoterol analogues by any effective route. Exemplary routes of administration include, but are not limited to, injection (such as subcutaneous, intramuscular, intradermal, intraperitoneal (“IP”), and intravenous (“IV”)), oral, sublingual, rectal, transdermal, intranasal, vaginal and inhalation routes.

Alkoxy: A radical (or substituent) having the structure —O—R, where R is a substituted or unsubstituted alkyl. Methoxy (—OCH₃) is an exemplary alkoxy group. In a substituted alkoxy, R is alkyl substituted with a non-interfering substituent. “Thioalkoxy” refers to —S—R, where R is substituted or unsubstituted alkyl. “Haloalkyloxy” means a radical —OR where R is a haloalkyl.

Alkoxy carbonyl: A group of the formula —C(O)OR, where R may be an optionally substituted alkyl or optionally substituted aryl. “Lower alkoxy carbonyl” groups are those where R contains from 1 to 10 (such as from 1 to 6) carbon atoms.

Alkyl: An acyclic, saturated, branched- or straight-chain hydrocarbon radical, which, unless expressly stated otherwise, contains from one to fifteen carbon atoms; for example, from one to ten, from one to six, or from one to four carbon atoms. This term includes, for example, groups such as methyl, ethyl, n-propyl, isopropyl, isobutyl, t-butyl, pentyl, heptyl, octyl, nonyl, decyl, or dodecyl. The term “lower alkyl” refers to an alkyl group containing from one to ten carbon atoms. Unless expressly referred to as an “unsubstituted alkyl,” alkyl groups can either be unsubstituted or substituted. An alkyl group can be substituted with one or more substituents (for example, up to two substituents for each methylene carbon in an alkyl chain). Exemplary alkyl substituents include, for instance, amino groups, amide, sulfonamide, halogen, cyano, carboxy, hydroxy, mercapto, trifluoromethyl, alkyl, alkoxy (such as methoxy), alkylthio, thioalkoxy, arylalkyl, heteroaryl, alkylamino, dialkylamino, alkylsulfano, keto, or other functionality.

Amino carbonyl (carbamoyl): A group of the formula C(O)N(R)R′, wherein R and R′ are independently of each other hydrogen or a lower alkyl group.

β2-adrenergic receptor (β2-AR): A subtype of adrenergic receptors that are members of the G-protein coupled receptor family. β2-AR subtype is involved in respiratory diseases, cardiovascular diseases, premature labor and, as disclosed herein, tumor development. Increased expression of β2-ARs can serve as therapeutic targets.

Cannabinoid Receptors: A class of cell membrane receptors under the G protein-coupled receptor superfamily. The cannabinoid receptors contain seven transmembrane spanning domains. Cannabinoid receptors are activated by three major groups of ligands, endocannabinoids (produced by the mammalian body), plant cannabinoids (such as THC, produced by the cannabis plant) and synthetic cannabinoids (such as HU-210). All of the endocannabinoids and plant cannabinoids are lipophilic, i.e., fat soluble, compounds. Two subtypes of cannabinoid receptors are CB₁ (see GenBank Accession No. NM_033181 mRNA and UniProt P21554, each of which is hereby incorporated by reference as of May 23, 2012) and CB₂ (see GenBank Accession No. NM_001841 mRNA and UniProt P34972, each of which is hereby incorporated by reference as of May 23, 2012). The CB₂ receptor is expressed mainly in the immune system and in hematopoietic cells. Additional non-CB₁ and non-CB₂ include GPR55 (GenBank Accession No. NM_005683.3 or NP_005674.2 protein, each of which is hereby incorporated by reference as of May 23, 2012), GPR119 (GenBank Accession No. NM_178471.2 or NP_848566.1 protein, each of which is hereby incorporated by reference as of May 23, 2012) and GPR18 (also known as N-arachidonyl glycine receptor and involved in microglial migration, GenBank Accession No. NM_001098200 mRNA, NP_001091670.1, each of which is hereby incorporated by reference as of May 23, 2012).

The protein sequences of CB₁ and CB₂ receptors are about 44% similar. When only the transmembrane regions of the receptors are considered, amino acid similarity between the two receptor subtypes is approximately 68%. In addition, minor variations in each receptor have been identified. Cannabinoids bind reversibly and stereo-selectively to the cannabinoid receptors. The affinity of an individual cannabinoid to each receptor determines the effect of that cannabinoid. Cannabinoids that bind more selectively to certain receptors are more desirable for medical usage. GPR55 is coupled to the G-protein G₁₃ and/or Gn and activation of the receptor leads to stimulation of rhoA, cdc42 and racl. GPR55 is activated by the plant cannabinoids A⁹-THC and cannabidiol, and the endocannabinoids anandamide, 2-AG, noladin ether in the low nanomolar range. In contrast, CB₁ and CB₂ receptors are coupled to inhibitory G proteins. This indicates that both types of receptors will have different readouts. For example, activation of CB₁ causes apoptosis whereas increase in GPR55 activity is oncogenic. The CB₁ receptor antagonist (also termed ‘inverse agonist’) compound, AM251, is, in fact, an agonist for GPR55. It binds GPR55 and is readily internalized. This illustrates the opposite behavior of these two GPCRs.

Carbamate: A group of the formula —OC(O)N(R)—, wherein R is H, or an aliphatic group, such as a lower alkyl group or an aralkyl group.

Chemotherapy; chemotherapeutic agents: As used herein, any chemical agent with therapeutic usefulness in the treatment of diseases characterized by abnormal cell growth. Such diseases include tumors, neoplasms, and cancer as well as diseases characterized by hyperplastic growth. In one embodiment, a chemotherapeutic agent is an agent of use in treating neoplasms such as solid tumors, including a tumor associated with CB receptor activity and/or expression. In embodiments, a chemotherapeutic agent is radioactive molecule. In embodiments, a CB receptor regulator, such as one or more fenoterol analogues or a combination thereof is a chemotherapeutic agent. In one example, a chemotherapeutic agent is carmustine, lomustine, procarbazine, streptozocin, or a combination thereof. One of skill in the art can readily identify a chemotherapeutic agent of use (e.g., see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R. (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

Control or Reference Value: A “control” refers to a sample or standard used for comparison with a test sample. In some embodiments, the control is a sample obtained from a healthy subject or a tissue sample obtained from a patient diagnosed with a disorder or disease, such as a tumor, that did not respond to treatment with a p2-agonist. In some embodiments, the control is a historical control or standard reference value or range of values.

Derivative: A chemical substance that differs from another chemical substance by one or more functional groups. In embodiments, a derivative retains a biological activity of a molecule from which it was derived.

Effective amount: An amount of agent that is sufficient to generate a desired response, such as reducing or inhibiting one or more signs or symptoms associated with a condition or disease. When administered to a subject, a dosage will generally be used that will achieve target tissue concentrations. In some examples, an “effective amount” is one that treats one or more symptoms and/or underlying causes of any of a disorder or disease. In some examples, an “effective amount” is a “therapeutically effective amount” in which the agent alone with an additional therapeutic agent(s) (for example a chemotherapeutic agent) induces the desired response such as treatment of a tumor. In one example, a desired response is to decrease tumor size or metastasis in a subject to whom the therapy is administered. Tumor metastasis does not need to be completely eliminated for the composition to be effective. For example, a composition can decrease metastasis by a desired amount, for example by at least 20%, at least 50%, at least 60%, at least 70%, at least 80%, at least 90%, at least 95%, at least 98%, or even at least 100% (elimination of the tumor), as compared to metastasis in the absence of the composition.

In particular examples, it is an amount of an agent effective to decrease a number of carcinoma cells, such as in a subject to whom it is administered, for example a subject having one or more carcinomas. The cancer cells do not need to be completely eliminated for the composition to be effective. For example, a composition can decrease the number of cancer cells by a desired amount, for example by at least 20%, at least 50%, at least 60%, at least 70%, at least 80%, at least 90%, at least 95%, at least 98%, or even at least 100% (elimination of detectable cancer cells), as compared to the number of cancer cells in the absence of the composition.

The effective amount of a composition useful for reducing, inhibiting, and/or treating a disorder in a subject will be dependent on the subject being treated, the severity of the disorder, and the manner of administration of the therapeutic composition. Effective amounts a therapeutic agent can be determined in many different ways, such as assaying for a reduction in tumor size or improvement of physiological condition of a subject having a tumor, such as a brain tumor. Effective amounts also can be determined through various in vitro, in vivo or in situ assays.

Fenoterol Analogues: Fenoterol analogues include (R,R′)-4′-methoxy-1-naphthylfenoterol (“MNF”), (R,S′)-4′-methoxy-1-naphthylfenoterol, (R,R′)-ethylMNF, (R,R′)-napthylfenoterol, (R,S′)-napthylfenoterol, (R,R′)-ethyl-naphthylfenoterol, (R,R′)-4′-amino-1-naphthylfenoterol, (R,R′)-4′-hydroxy-1-naphthylfenoterol, (R,R′)-4′-methoxy-ethylfenoterol, (R,R′)-methoxyfenoterol, (R,R′)-ethylfenoterol, (R,R′)-fenoterol and their respective stereoisomers.

Inflammation: When damage to tissue occurs, the body's response to the damage is usually inflammation. The damage may be due to trauma, lack of blood supply, hemorrhage, autoimmune attack, transplanted exogenous tissue or infection. This generalized response by the body includes the release of many components of the immune system (for instance, IL-1 and TNF), attraction of cells to the site of the damage, swelling of tissue due to the release of fluid and other processes.

Isomers: Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that contain two or more chiral centers and are not mirror images of one another are termed “diastereomers.” Steroisomers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−) isomers, respectively). A chiral compound can exist as either an individual enantiomer or as a mixture thereof. A mixture containing equal proportions of the enantiomers is called a “racemic mixture.”

The compounds described herein may possess one or more asymmetric centers; such compounds can therefore be produced as individual (R), (S), (R,R′), (R,S′), (S,R′) and (S,S′)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well known in the art (see, e.g., March, Advanced Organic Chemistry, 4th edition, New York: John Wiley and Sons, 1992, Chapter 4).

Optional: “Optional” or “optionally” means that the subsequently described event or circumstance can but need not occur, and that the description includes instances where said event or circumstance occurs and instances where it does not.

Pharmaceutically Acceptable Carriers: The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds or molecules, such as one or more nucleic acid molecules, proteins or antibodies that bind these proteins, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phenyl: Phenyl groups may be unsubstituted or substituted with one, two or three substituents, with substituent(s) independently selected from alkyl, heteroalkyl, aliphatic, heteroaliphatic, thioalkoxy, halo, haloalkyl (such as —CF₃), nitro, cyano, —OR (where R is hydrogen or alkyl), —N(R)R′ (where R and R′ are independently of each other hydrogen or alkyl), —COOR (where R is hydrogen or alkyl) or —C(O)N(R′)R″ (where R′ and R″ are independently selected from hydrogen or alkyl).

Purified: The term “purified” does not require absolute purity; rather, it is intended as a relative term. Thus, for example, a purified preparation is one in which a desired component such as an (R,R′)-enantiomer of fenoterol is more enriched than it was in a preceding environment such as in a (+)-fenoterol mixture. A desired component such as (R,R′)-enantiomer of fenoterol is considered to be purified, for example, when at least about 70%, 80%, 85%, 90%, 92%, 95%, 97%, 98%, or 99% of a sample by weight is composed of the desired component. Purity of a compound may be determined, for example, by high performance liquid chromatography (HPLC) or other conventional methods. In an example, the fenoterol analogue enantiomers are purified to represent greater than 90%, often greater than 95% of the other enantiomers present in a purified preparation. In other cases, the purified preparation may be essentially homogeneous, wherein other stereoisomers are less than 1%.

Compounds described herein may be obtained in a purified form or purified by any of the means known in the art, including silica gel and/or alumina chromatography. See, e.g., Introduction to Modern Liquid Chromatography, 2nd Edition, ed. by Snyder and Kirkland, New York: John Wiley and Sons, 1979; and Thin Layer Chromatography, ed. by Stahl, New York: Springer Verlag, 1969. In an example, a compound includes purified fenoterol or fenoterol analogue with a purity of at least about 70%, 80%, 85%, 90%, 92%, 95%, 97%, 98%, or 99% of a sample by weight relative to other contaminants. In a further example, a compound includes at least two purified stereoisomers each with a purity of at least about 70%, 80%, 85%, 90%, 92%, 95%, 97%, 98%, or 99% of a sample by weight relative to other contaminants. For instance, a compound can include a substantially purified (R,R′)-fenoterol analogue and a substantially purified (R,S′)-fenoterol analogue.

Subject: The term “subject” includes both human and veterinary subjects, for example, humans, non-human primates, dogs, cats, horses, rats, mice, and cows. Similarly, the term mammal includes both human and non-human mammals.

Tissue: A plurality of functionally related cells. A tissue can be a suspension, a semi-solid, or solid. Tissue includes cells collected from a subject such as the brain or a portion thereof.

Tumor: All neoplastic cell growth and proliferation, whether malignant or benign, and all pre-cancerous and cancerous cells and tissues. A primary tumor is tumor growing at the anatomical site where tumor progression began and proceeded to yield this mass.

Under conditions sufficient for: A phrase that is used to describe any environment that permits the desired activity. In one example, under conditions sufficient for includes administering one or more fenoterol analogues to a subject to at a concentration sufficient to allow the desired activity. In some examples, the desired activity is reducing or inhibiting a sign or symptom associated with a disorder or disease, such as a breast or pancreatic, can be evidenced, for example, by a delayed onset of clinical symptoms of the tumor in a susceptible subject, a reduction in severity of some or all clinical symptoms of the tumor, a slower progression of the tumor (for example by prolonging the life of a subject having the tumor), a reduction in the number of tumor reoccurrence, an improvement in the overall health or well-being of the subject, or by other parameters well known in the art that are specific to the particular disease. In one particulate example, the desired activity is preventing or inhibiting tumor growth, such as breast cancer or pancreatic cancer growth. Tumor growth does not need to be completely inhibited for the treatment to be considered effective. For example, a partial reduction or slowing of growth such as at least about a 10% reduction, such as at least 20%, at least about 30%, at least about 40%, at least about 50% or greater is considered to be effective.

### Chemical Structure of Fenoterol Analogues

Fenoterol analogues useful in the methods herein include (R,R′)-4′-methoxy-1-naphthylfenoterol (“MNF”), (R,S′)-4′-methoxy-1-naphthylfenoterol, (R,R′)-ethylMNF, (R,R′)-napthylfenoterol, (R,S′)-napthylfenoterol, (R,R′)-ethyl-naphthylfenoterol, (R,R′)-4′-amino-1-naphthylfenoterol, (R,R′)-4′-hydroxy-1-naphthylfenoterol, (R,R′)-4′-methoxy-ethylfenoterol, (R,R′)-4′-methoxyfenoterol, (R,R′)-ethylfenoterol, (R,R′)-fenoterol and their respective stereoisomers.

Examples of suitable groups for R₁-R₃ that can be cleaved in vivo to provide a hydroxy group include, without limitation, acyl, acyloxy and alkoxy carbonyl groups. Compounds having such cleavable groups are referred to as “prodrugs.” The term “prodrug,” as used herein, means a compound that includes a substituent that is convertible in vivo (e.g., by hydrolysis) to a hydroxyl group. Various forms of prodrugs are known in the art, for example, as discussed in Bundgaard, (ed.), Design of Prodrugs, Elsevier (1985); Widder, et al. (ed.), Methods in Enzymology, Vol. 4, Academic Press (1985); Krogsgaard-Larsen, et al., (ed), Design and Application of Prodrugs, Textbook of Drug Design and Development, Chapter 5, 113 191 (1991); Bundgaard, et al., Journal of Drug Delivery Reviews, 8: 1 38 (1992); Bundgaard, Pharmaceutical Sciences, 77:285 et seq. (1988); and Higuchi and Stella (eds.) Prodrugs as Novel Drug Delivery Systems, American Chemical Society (1975).

In embodiments, administering comprises administering a therapeutically effective amount of MNF, NF or a combination thereof. In some embodiments, administering comprises administering a therapeutically effective amount of MNF.

Particular method embodiments contemplate the use of solvates (such as hydrates), pharmaceutically acceptable salts and/or different physical forms of the fenoterol analogues herein described.

### Solvates, Salts and Physical Forms

“Solvate” means a physical association of a compound with one or more solvent molecules. This physical association involves varying degrees of ionic and covalent bonding, including by way of example covalent adducts and hydrogen bonded solvates. In certain instances the solvate will be capable of isolation, for example when one or more solvent molecules are incorporated in the crystal lattice of the crystalline solid. “Solvate” encompasses both solution-phase and isolable solvates. Representative solvates include ethanol-associated compound, methanol-associated compounds, and the like. “Hydrate” is a solvate wherein the solvent molecule(s) is/are H₂O.

The disclosed compounds also encompass salts including, if several salt-forming groups are present, mixed salts and/or internal salts. The salts are generally pharmaceutically acceptable salts that are non-toxic. Salts may be of any type (both organic and inorganic), such as fumarates, hydrobromides, hydrochlorides, sulfates and phosphates. In an example, salts include non-metals (e.g., halogens) that form group VII in the periodic table of elements. For example, compounds may be provided as a hydrobromide salt.

Additional examples of salt-forming groups include, but are not limited to, a carboxyl group, a phosphonic acid group or a boronic acid group, that can form salts with suitable bases. These salts can include, for example, nontoxic metal cations, which are derived from metals of groups IA, IB, IIA and IIB of the periodic table of the elements. In one embodiment, alkali metal cations such as lithium, sodium or potassium ions, or alkaline earth metal cations such as magnesium or calcium ions can be used. The salt can also be a zinc or an ammonium cation. The salt can also be formed with suitable organic amines, such as unsubstituted or hydroxyl-substituted mono-, di- or tri-alkylamines, in particular mono-, di- or tri-alkylamines, or with quaternary ammonium compounds, for example with N-methyl-N-ethylamine, diethylamine, triethylamine, mono-, bis- or tris-(2-hydroxy-lower alkyl)amines, such as mono-, bis- or tris-(2-hydroxyethyl)amine, 2-hydroxy-tert-butylamine or tris(hydroxymethyl)methylamine, N,N-di-lower alkyl-N-(hydroxy-lower alkyl)amines, such as N,N-dimethyl-N-(2-hydroxyethyl)amine or tri-(2-hydroxyethyl)amine, or N-methyl-D-glucamine, or quaternary ammonium compounds such as tetrabutylammonium salts.

Exemplary compounds disclosed herein possess at least one basic group that can form acid-base salts with inorganic acids. Examples of basic groups include, but are not limited to, an amino group or imino group. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, and, in addition, with amino acids, for example with a-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid. In a currently preferred embodiment, fenoterol is provided as a hydrobromide salt and exemplary fenoterol analogues are provided as their fumarate salts.

Additional counterions for forming pharmaceutically acceptable salts are found in Remington's Pharmaceutical Sciences, 19th Edition, Mack Publishing Company, Easton, Pa., 1995. In one aspect, employing a pharmaceutically acceptable salt may also serve to adjust the osmotic pressure of a composition.

In certain embodiments the compounds used in the method are provided are polymorphous. As such, the compounds can be provided in two or more physical forms, such as different crystal forms, crystalline, liquid crystalline or non-crystalline (amorphous) forms.

### Use for the Manufacture of a Medicament

Any of the above described compounds (e.g., (R,R′) and/or (R,S′) fenoterol analogues or a hydrate or pharmaceutically acceptable salt thereof) or combinations thereof are intended for use in the manufacture of a medicament for treatment of pancreatic cancer.

Formulations suitable for such medicaments, subjects who may benefit from same and other related features are described elsewhere herein.

### Methods of Synthesis

The disclosed fenoterol analogues can be synthesized by any method known in the art including those described in U.S. Patent Application Publication No. US 2010-0168245 A1, U.S. Patent Application Publication No. US 2012-0157543 A1 and International Patent Publication No. WO 2011/112867, each of which is hereby incorporated by reference in its entirety. Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley—Interscience, 2001; or Vogel, A Textbook of Practical Organic Chemistry, Including Qualitative Organic Analysis, Fourth Edition, New York: Longman, 1978).

Compounds as described herein may be purified by any of the means known in the art, including chromatographic means, such as HPLC (including chiral HPLC), preparative thin layer chromatography, flash column chromatography and ion exchange chromatography. Any suitable stationary phase can be used, including normal and reversed phases as well as ionic resins. Most typically the disclosed compounds are purified via open column chromatography or prep chromatography. Suitable exemplary syntheses of fenoterol analogues are provided below:

Scheme I: An exemplary synthesis of 4 stereoisomers of 1-6 including the coupling of the epoxide formed from either (R)- or (S)-3′,5′-dibenzyloxyphenyl bromohydrin with the (R)- or (S)-enantiomer of the appropriate benzyl-protected 2-amino-3-benzylpropane (1-5) or the (R)- or (S)-enantiomer of N-benzyl-2-aminoheptane (6).

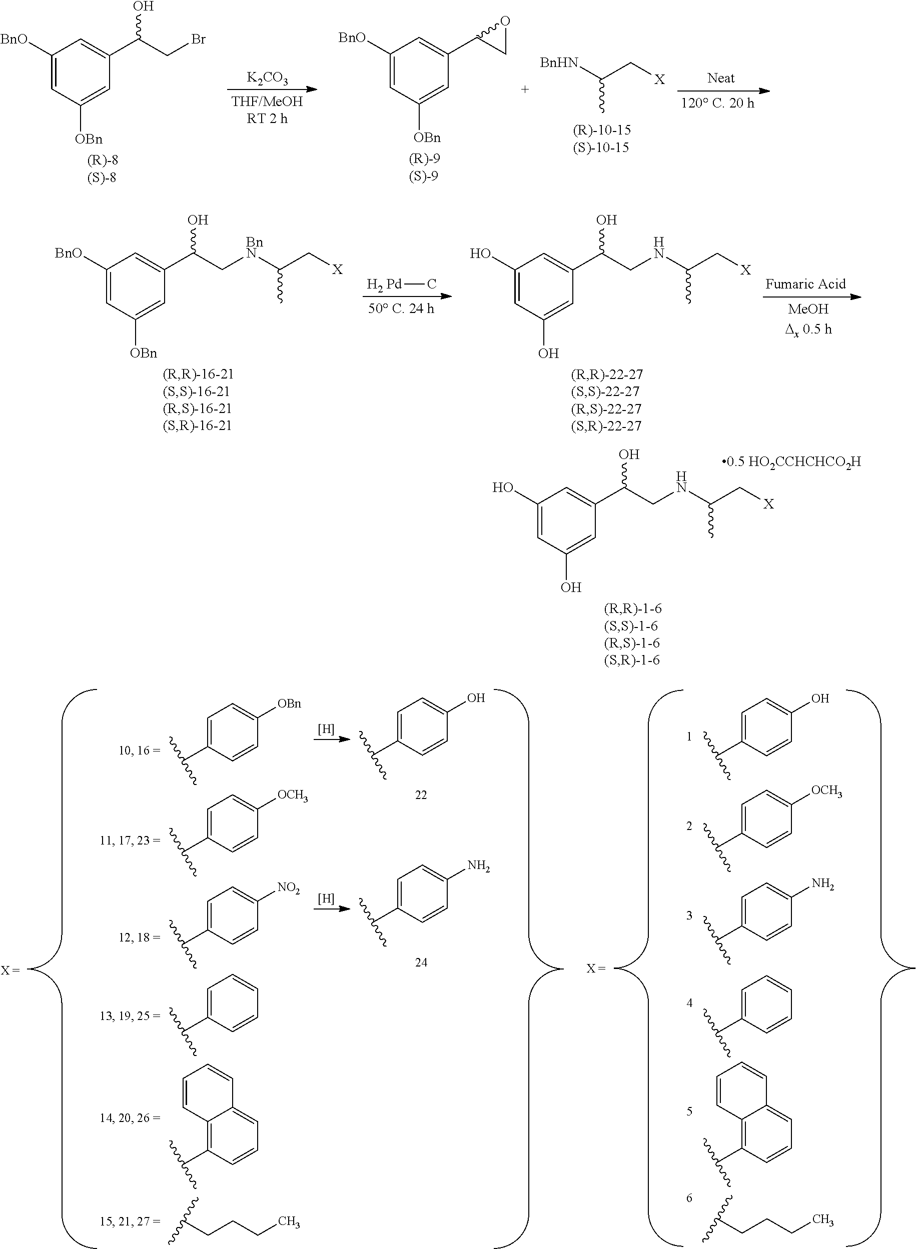

Scheme II: Exemplary synthesis of (R)-7 and (S)-7 using 2-phenethylamine. The resulting compounds may be deprotected by hydrogenation over Pd/C and purified as the fumarate salts.

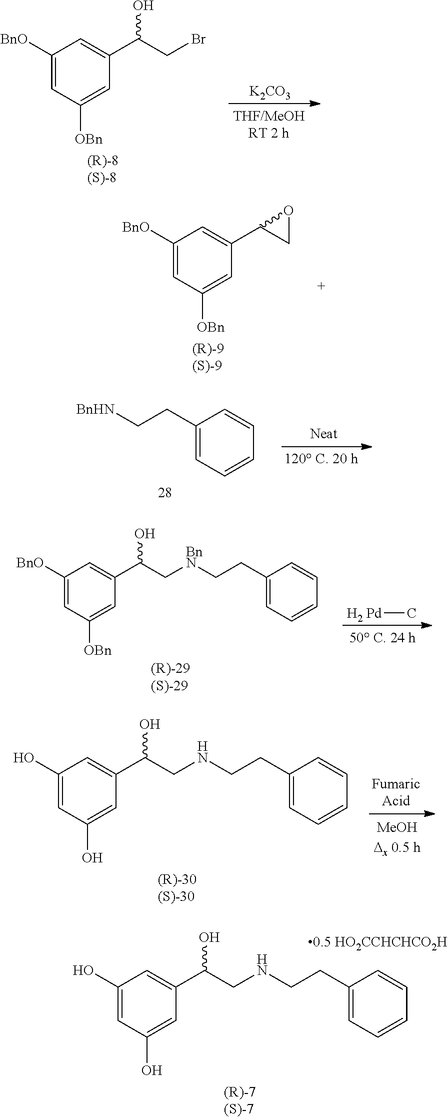

Scheme III describes an exemplary synthesis for the chiral building blocks used in Scheme II. The (R)- and (S)-3′,5′-dibenzyloxyphenyl-bromohydrin enantiomers were obtained by the enantio specific reduction of 3,5-dibenzyloxy-a-bromoacetophenone using boron-methyl sulfide complex (BH₃SCH₃) and either (1R,2S)- or (1S,2R)-cis-1-amino-2-indanol. The required (R)- and (S)-2-benzylaminopropanes were prepared by enantioselective crystallization of the rac-2-benzylaminopropanes using either (R)- or (S)-mandelic acid as the counter ion.

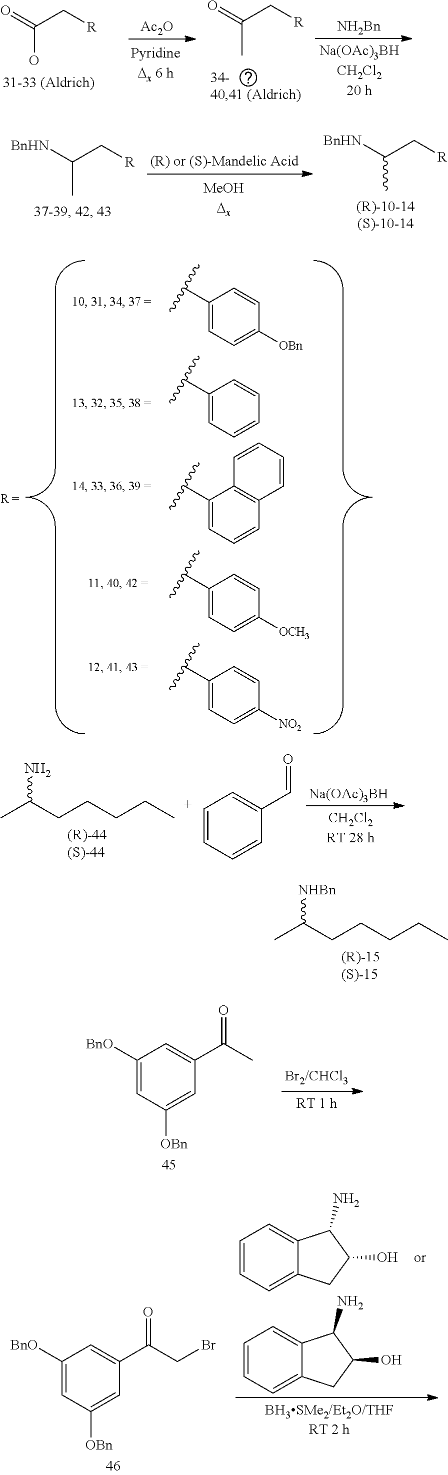

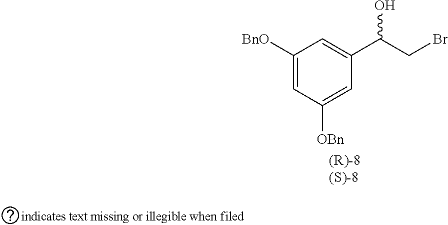 [SMILES: OC(CBr)c1cc(OCc2ccccc2)cc(OCc2ccccc2)c1]

### Pharmaceutical Compositions

The disclosed fenoterol analogues can be useful, at least, for reducing or inhibiting one or more symptoms or signs associated with cancer. Accordingly, pharmaceutical compositions comprising at least one disclosed fenoterol analogue are also described herein.

Formulations for pharmaceutical compositions are well known in the art. For example, Remington's Pharmaceutical Sciences, by E. W. Martin, Mack Publishing Co., Easton, Pa., 19th Edition, 1995, describes exemplary formulations (and components thereof) suitable for pharmaceutical delivery of (R,R′)-fenoterol and disclosed fenoterol analogues. Pharmaceutical compositions comprising at least one of these compounds can be formulated for use in human or veterinary medicine. Particular formulations of a disclosed pharmaceutical composition may depend, for example, on the mode of administration (e.g., oral or parenteral) and/or on the disorder to be treated. In some embodiments, formulations include a pharmaceutically acceptable carrier in addition to at least one active ingredient, such as a fenoterol compound.

Pharmaceutically acceptable carriers useful for the disclosed methods and compositions are conventional in the art. The nature of a pharmaceutical carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle.

For solid compositions such as powder, pill, tablet, or capsule forms conventional non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances or excipients, such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations.

The disclosed pharmaceutical compositions may be formulated as a pharmaceutically acceptable salt. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydriodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Lists of other suitable pharmaceutically acceptable salts are found in Remington's Pharmaceutical Sciences, 19th Edition, Mack Publishing Company, Easton, Pa., 1995. A pharmaceutically acceptable salt may also serve to adjust the osmotic pressure of the composition.

The dosage form of a disclosed pharmaceutical composition will be determined by the mode of administration chosen. For example, in addition to injectable fluids, oral dosage forms may be employed. Oral formulations may be liquid such as syrups, solutions or suspensions or solid such as powders, pills, tablets, or capsules. Methods of preparing such dosage forms are known, or will be apparent, to those skilled in the art.

Certain embodiments of the pharmaceutical compositions comprising a disclosed compound may be formulated in unit dosage form suitable for individual administration of precise dosages. The amount of active ingredient such as (R,R′)-MNF or NF administered will depend on the subject being treated, the severity of the disorder, and the manner of administration, and is known to those skilled in the art. Within these bounds, the formulation to be administered will contain a quantity of the extracts or compounds disclosed herein in an amount effective to achieve the desired effect in the subject being treated.

In particular examples, for oral administration the compositions are provided in the form of a tablet containing from about 1.0 to about 50 mg of the active ingredient, particularly about 2.0 mg, about 2.5 mg, 5 mg, about 10 mg, or about 50 mg of the active ingredient for the symptomatic adjustment of the dosage to the subject being treated. In one exemplary oral dosage regimen, a tablet containing from about 1 mg to about 50 mg (such as about 2 mg to about 10 mg) active ingredient is administered two to four times a day, such as two times, three times or four times.

In other examples, a suitable dose for parental administration is about 1 milligram per kilogram (mg/kg) to about 100 mg/kg, such as a dose of about 10 mg/kg to about 80 mg/kg, such including about 1 mg/kg, about 2 mg/kg, about 5 mg/kg, about 20 mg/kg, about 30 mg/kg, about 40 mg/kg, about 50 mg/kg, about 80 mg/kg or about 100 mg/kg administered parenterally. However, other higher or lower dosages also could be used, such as from about 0.001 mg/kg to about 1 g/kg, such as about 0.1 to about 500 mg/kg, including about 0.5 mg/kg to about 200 mg/kg.

Single or multiple administrations of the composition comprising one or more of the disclosed compositions can be carried out with dose levels and pattern being selected by the treating physician. Generally, multiple doses are administered. In a particular example, the composition is administered parenterally once per day. However, the composition can be administered twice per day, three times per day, four times per day, six times per day, every other day, twice a week, weekly, or monthly. Treatment will typically continue for at least a month, more often for two or three months, sometimes for six months or a year, and may even continue indefinitely, i.e., chronically. Repeat courses of treatment are also possible.

In embodiments, the pharmaceutical composition is administered without concurrent administration of a second agent for the treatment of cancer. In one specific, non-limiting example, one or more of the disclosed compositions is administered without concurrent administration of other agents, such as without concurrent administration of an additional agent also known to target the tumor. In other specific non-limiting examples, a therapeutically effective amount of a disclosed pharmaceutical composition is administered concurrently with an additional agent, including an additional therapy. For example, the disclosed compounds are administered in combination with a chemotherapeutic agent, anti-oxidants, anti-inflammatory drugs or combinations thereof.

In other examples, a disclosed pharmaceutical composition is administered as adjuvant therapy. For example, a pharmaceutical composition containing one or more of the disclosed compounds is administered orally daily to a subject in order to prevent or retard tumor growth. In one particular example, a composition containing equal portions of two or more disclosed compounds is provided to a subject. In one example, a composition containing unequal portions of two or more disclosed compounds is provided to the subject. For example, a composition contains unequal portions of a (R,R′)-fenoterol derivative and a (S,R′)-fenoterol derivative and/or a (R,S′)-derivative. In one particular example, the composition includes a greater amount of the (S,R′)- or (R,S′)-fenoterol derivative. Such therapy can be given to a subject for an indefinite period of time to inhibit, prevent, or reduce tumor reoccurrence.

### Methods of Use

The present disclosure includes methods of treating disorders including reducing or inhibiting one or more signs or symptoms associated with cancer, such as pancreatic cancer. Disclosed methods include administering fenoterol, such as (R,R′)-fenoterol, a disclosed fenoterol analogue or a combination thereof (and, optionally, one or more other pharmaceutical agents) depending upon the receptor population of the tumor, to a subject in a pharmaceutically acceptable carrier and in an amount effective to suppress multidrug resistance to chemotherapeutic agents in cancer cells, such as pancreatic cancer cells. Treatment of a tumor includes preventing or reducing signs or symptoms associated with the presence of such tumor (for example, by reducing the size or volume of the tumor or a metastasis thereof). Such reduced growth can in some examples decrease or slow metastasis of the tumor, or reduce the size or volume of the tumor by at least 10%, at least 20%, at least 50%, or at least 75%, such as between 10%-90%, 20%-80%, 30%-70%, 40%-60%, including a 10%, 15%, 20%, 25%, 30%, 35%, 40%, 45%, 50%, 55%, 60%, 65%, 70%, 75%, 80%, 90%, or 95% reduction. In another example, treatment includes reducing the invasive activity of the tumor in the subject, for example by reducing the ability of the tumor to metastasize. In some examples, treatment using the methods disclosed herein prolongs the time of survival of the subject.

Routes of administration useful in the disclosed methods include but are not limited to oral and parenteral routes, such as intravenous (IV), intraperitoneal (IP), rectal, topical, ophthalmic, nasal, and transdermal as described in detail above.

An effective amount of a disclosed fenoterol analogue will depend, at least, on the particular method of use, the subject being treated, the severity of the tumor, and the manner of administration of the therapeutic composition. A “therapeutically effective amount” of a composition is a quantity of a specified compound sufficient to achieve a desired effect in a subject being treated. For example, this may be the amount of a fenoterol analogue necessary to prevent or inhibit tumor growth and/or one or more symptoms associated with the tumor in a subject. Ideally, a therapeutically effective amount of a disclosed fenoterol analogue is an amount sufficient to prevent or inhibit a tumor, such as a brain or liver tumor growth and/or one or more symptoms associated with the tumor in a subject without causing a substantial cytotoxic effect on host cells. In embodiments, a therapeutically effective amount may be an amount sufficient to suppress multidrug resistance in tumor cells.

Therapeutically effective doses of a disclosed fenoterol compound or pharmaceutical composition can be determined by one of skill in the art, with a goal of achieving concentrations that are at least as high as the IC₅₀ of the applicable compound disclosed in the examples herein. An example of a dosage range is from about 0.001 to about 10 mg/kg body weight orally in single or divided doses. In particular examples, a dosage range is from about 0.005 to about 5 mg/kg body weight orally in single or divided doses (assuming an average body weight of approximately 70 kg; values adjusted accordingly for persons weighing more or less than average). For oral administration, the compositions are, for example, provided in the form of a tablet containing from about 1.0 to about 50 mg of the active ingredient, particularly about 2.5 mg, about 5 mg, about 10 mg, or about 50 mg of the active ingredient for the symptomatic adjustment of the dosage to the subject being treated. In one exemplary oral dosage regimen, a tablet containing from about 1 mg to about 50 mg active ingredient is administered two to four times a day, such as two times, three times or four times.

In other examples, a suitable dose for parental administration is about 1 milligram per kilogram (mg/kg) to about 100 mg/kg, such as a dose of about 10 mg/kg to about 80 mg/kg, such including about 1 mg/kg, about 2 mg/kg, about 5 mg/kg, about 20 mg/kg, about 30 mg/kg, about 40 mg/kg, about 50 mg/kg, about 80 mg/kg or about 100 mg/kg administered parenterally. However, other higher or lower dosages also could be used, such as from about 0.001 mg/kg to about 1 g/kg, such as about 0.1 to about 500 mg/kg, including about 0.5 mg/kg to about 200 mg/kg.

Single or multiple administrations of the composition comprising one or more of the disclosed compositions can be carried out with dose levels and pattern being selected by the treating physician. Generally, multiple doses are administered. In a particular example, the composition is administered parenterally once per day. However, the composition can be administered twice per day, three times per day, four times per day, six times per day, every other day, twice a week, weekly, or monthly. Treatment will typically continue for at least a month, more often for two or three months, sometimes for six months or a year, and may even continue indefinitely, i.e., chronically. Repeat courses of treatment are also possible.

The specific dose level and frequency of dosage for any particular subject may be varied and will depend upon a variety of factors, including the activity of the specific compound, the metabolic stability and length of action of that compound, the age, body weight, general health, sex and diet of the subject, mode and time of administration, rate of excretion, drug combination, and severity of the condition of the subject undergoing therapy.

### Selecting a Subject

Subjects can be screened prior to initiating the disclosed therapies, for example to select a subject in need of cancer treatment or at risk of developing cancer. Briefly, the method can include screening subjects to determine if they have or are at risk of developing cancer, such as if the subject is in need of cancer inhibition. In exemplary embodiments, a subject in need of the disclosed therapies is selected by detecting a tumor expressing cannabinoid (CB) receptor (including but not limited to GPR55) or regulated by cannabinoid (CB) receptor (including but not limited to GPR55) activity or expression in a sample obtained from a subject identified as having, suspected of having or at risk of acquiring such a tumor. For example, detection of altered, such as at least a 10% alteration, including a 10%-90%, 20%-80%, 30%-70%, 40%-60%, such as a 10%, 15%, 20%, 25%, 30%, 35%, 40%, 45%, 50%, 55%, 60%, 65%, 70%, 75%, 80%, 90%, 95% alteration or more in cannabinoid (CB) receptor (including but not limited to GPR55) expression or activity as compared to cannabinoid (CB) receptor (including but not limited to GPR55) expression or activity in the absence of a primary tumor, indicates that the tumor can be treated using the compositions and methods provided herein.

In embodiments, subjects can be screened prior to initiating the disclosed therapies, for example to select a subject having a tumor that includes cells that have developed a resistance to one or more chemotherapeutic agents. Such screening may, in embodiments, include genotype testing of the tumor to determine whether the tumor exhibits increased expression or overexpression of the multiple drug resistant genes. Genotype testing may be conducted either before or after treatment, either with a chemotherapeutic agent or in accordance with a method of the present disclosure. Another example of such screening may include performing immunihisto chemistry on cells of the tumor to detect expression of MDR proteins. For example, a biopsy sample can be tested to determine the presence of the MDR proteins on the tumor sample. Other techniques for screening to select a subject having a tumor that includes cells that have developed a resistance to one or more chemotherapeutic agents will be apparent to those skilled in the art reading this disclosure.

Pre-screening is not required prior to administration of the therapeutic agents disclosed herein (such as those including fenoterol, a fenoterol analogue or a combination thereof). In embodiments, the recurrence of a tumor or relapse may be used to infer that a subject has a tumor that includes cells that have developed a resistance to one or more chemotherapeutic agents, particularly to a chemotherapeutic agent that had been administered to the subject prior to the recurrence or relapse.

### Assessment

Following the administration of one or more therapies, subjects can be monitored for decreases in tumor growth, tumor volume or in one or more clinical symptoms associated with the tumor. In particular examples, subjects are analyzed one or more times, starting 7 days following treatment. Subjects can be monitored using any method known in the art including those described herein including imaging analysis.

### Additional Treatments and Additional Therapeutic Agents

In particular examples, if subjects are stable or have a minor, mixed or partial response to treatment, they can be re-treated after re-evaluation with the same schedule and preparation of agents that they previously received for the desired amount of time, including the duration of a subject's lifetime. A partial response is a reduction, such as at least a 10%, at least a 20%, at least a 30%, at least a 40%, at least a 50%, or at least a 70% reduction in one or more signs or symptoms associated with the disorder or disease, or activity, including tumor size or volume.

In some examples, the method further includes administering a therapeutic effective amount of a fenoterol analogue with additional therapeutic treatments. In particular examples, prior to, during, or following administration of a therapeutic amount of an agent that inhibits GPR55-mediated activation of cancer biomarkers in a tumor, the subject can receive one or more other therapies. In one example, the subject receives one or more treatments to remove or reduce the tumor prior to administration of a therapeutic amount of a composition including fenoterol, a fenoterol analogue or combination thereof.

Examples of such therapies include, but are not limited to, surgical treatment for removal or reduction of the tumor (such as surgical resection, cryotherapy, or chemoembolization), as well as anti-tumor pharmaceutical treatments which can include radiotherapeutic agents, anti-neoplastic chemotherapeutic agents, antibiotics, alkylating agents and antioxidants, kinase inhibitors, and other agents. Particular examples of additional therapeutic agents that can be used include microtubule-binding agents, DNA intercalators or cross-linkers, DNA synthesis inhibitors, DNA and/or RNA transcription inhibitors, antibodies, enzymes, enzyme inhibitors, and gene regulators. These agents (which are administered at a therapeutically effective amount) and treatments can be used alone or in combination. Methods and therapeutic dosages of such agents are known to those skilled in the art, and can be determined by a skilled clinician.

The present methods can include administration of the one or more therapeutic agents separately, sequentially, or concurrently, for example in a combined composition with a fenoterol analogue. In embodiments, the one or more therapeutic agents administered in addition to a fenoterol analogue is a chemotherapeutic agent. Chemotherapeutic agents known to be implicated by multidrug resistance that may be administered in accordance with the presently described methods include, but are not limited to, those drugs identified in Table 1.

TABLE 1
Illustrative examples of chemotherapeutic agents implicated by
multidrug resistance (MDR)

| | | MDR proteins clinically |
|---|---|---|
| Chemotherapeutic | Cancers against which drug is approved | implicated for |
| Agents | for treatment (FDA approved) | chemoreistence |
| Doxorubicin | Acute lymphoblastic leukemia, Acute | Pgp, BCRP, MRP1 |
| | myeloid leukemia, Breast cancer, Gastric | |
| | (stomach) cancer, Hodgkin lymphoma, | |
| | Neuroblastoma, Non-Hodgkin lymphoma, | |
| | Ovarian cancer, Small cell lung cancer, Soft | |
| | tissue and bone sarcomas, Thyroid cancer, | |
| | Transitional cell bladder cancer and Wilms | |
| | tumor. | |
| Daunorubicin | Acute lymphoblastic leukemia in adults and | Pgp, BCRP, MRP1 |
| | children, Acute myeloid leukemia in adults. | |
| Mitoxantrone | Acute myeloid leukemia (AML), Prostate | Pgp, BCRP, MRP1, LRP |
| | cancer. | |
| Paclitaxel | Breast cancer, Non-small cell lung cancer, | Pgp, BCRP, MRP1, |
| | AIDS-related Kaposi sarcoma, Ovarian | MRP2, LRP. |
| | cancer. | |
| Docetaxel | Breast cancer, Non-small cell lung cancer, | Pgp, BCRP, MRP1, |
| | Adenocarcinoma, Squamous cell carcinoma | MRP7 |
| | of the head and neck, Prostate cancer. | |
| Vincristine | Acute leukemia, Hodgkin lymphoma, | Pgp, MRP1 |
| | Neuroblastoma, Non-Hodgkin lymphoma | |
| | (NHL), Rhabdomyosarcoma, Wilms tumor. | |
| Vinblastine | Breast cancer, Choriocarcinoma, Hodgkin | Pgp, MRP1, MRP2 |
| | lymphoma, Kaposi sarcoma. | |
| | Mycosis fungoides, Non-Hodgkin lymphoma | |
| | (NHL), Testicular cancer. | |
| Cisplatin | Bladder cancer, Ovarian cancer, Testicular | Pgp, BCRP, MRP1, |
| | cancer | MRP2, MRP3 |
| Methotrexate | Acute lymphoblastic leukemia, Breast cancer, | Pgp, BCRP, MRP1, |
| | Gestational trophoblastic disease, Head and | MRP3, MRP4, MRP5 |
| | neck cancer (certain types), Lung cancer, | |
| | Mycosis fungoides, Non-Hodgkin lymphoma, | |
| | Osteosarcoma | |
| Gemcitabine | Breast cancer, Ovarian cancer, Pancreatic | Pgp, BCRP, MRP1, |
| | cancer, Non-small cell lung cancer | MRP2, MRP5 |
| 5-Fluorouracil | Breast cancer, Colorectal cancer, Gastric | Pgp, BCRP, MRP1, |
| | (stomach) cancer, Pancreatic cancer. | MRP5 |
| Bortezomib | Multiple Myeloma and Mantle cell lymphoma | Pgp, MRP1 |
| Carfilzomib | Multiple myeloma | Pgp |
| Erlotinib | Non-small cell lung cancer, Pancreatic cancer | Pgp, BCRP, and MRP2 |
| Ceritinib | Non-small cell lung cancer | Pgp |
| Sunitib | Gastrointestinal stromal tumor, Pancreatic | Pgp, BCRP |
| | cancer, Renal cell carcinoma | |
| Imatinib | Acute lymphoblastic leukemia, Chronic | Pgp, BCRP |
| | myelogenous leukemia, Dermatofibrosarcoma | |
| | protuberans, Gastrointestinal stromal tumor | |
| | (GIST), Myelodysplastic/myeloproliferative | |
| | neoplasms, Systemic mastocytosis. | |
| Irinotecan | Colorectal cancer, metastatic pancreatic | Pgp, BCRP, MRP1, |
| | cancer, neuroblastoma | MRP4 |
| Topotecan | Ovarian cancer, Small cell lung cancer, | Pgp, BCRP, MRP4 |
| | Cervical cancer | |
| Palbociclib | Breast cancer, glioblastoma (GBM) and | Pgp, BCRP |
| | diffuse intrinsic pontine gliomas (DIPG) | |
| Vemurafenib | Melanoma | Pgp, BCRP, MRP1 |

“Microtubule-binding agent” refers to an agent that interacts with tubulin to stabilize or destabilize microtubule formation thereby inhibiting cell division. Examples of microtubule-binding agents that can be used in conjunction with the disclosed therapy include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used and are known to those of ordinary skill in the art. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and/or 5,912,264 can be used.

The following classes of compounds may be of use in the methods described herein: DNA and/or RNA transcription regulators, including, without limitation, actinomycin D, daunorubicin, doxorubicin and derivatives and analogs thereof also are suitable for use in combination with the disclosed therapies; DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof; DNA synthesis inhibitors including, without limitation, methotrexate, 5-fluoro-5′-deoxyuridine, 5-fluorouracil and analogs thereof; enzyme inhibitors including, without limitation, camptothecin, etoposide, formestane, trichostatin and derivatives and analogs thereof alkylating agents including, without limitation, carmustine or lomustine); compounds that affect gene regulation include agents that result in increased or decreased expression of one or more genes, including, without limitation, raloxifene, 5-azacytidine, 5-aza-2′-deoxycytidine, tamoxifen, 4-hydroxytamoxifen, mifepristone and derivatives and analogs thereof; and kinase inhibitors including, without limitation, Gleevac, Iressa, and Tarceva that prevent phosphorylation and activation of growth factors.

Other therapeutic agents, for example anti-tumor agents, that may or may not fall under one or more of the classifications above, also are suitable for administration in combination with the disclosed therapies. By way of example, such agents include adriamycin, apigenin, rapamycin, zebularine, cimetidine, and derivatives and analogues thereof.

In embodiments, the present methods include administering two or more compounds that are GPR55 antagonists. In embodiments, a fenoterol analog is administered separately, sequentially, or concurrently with a second GPR55 antagonist, such as, for example, CID 16020046.

In one example, at least a portion of the tumor is surgically removed (for example via cryotherapy), irradiated, chemically treated (for example via chemoembolization) or combinations thereof, prior to administration of the disclosed therapies (such as administration of fenoterol, a fenoterol analogue or a combination thereof). For example, a subject can have at least a portion of the tumor surgically excised prior to administration of the disclosed therapies. In an example, one or more chemotherapeutic agents are administered following treatment with a composition including fenoterol, a fenoterol analogue or a combination thereof.

The subject matter of the present disclosure is further illustrated by the following non-limiting Examples.

### Materials and Methods

The material and methods used for the following Examples were as follows:

Materials.

(R,R′)-, (R,S′)-, (S,R)- and (S,S′)-fenoterol and the fenoterol analogs, (R,R′)-ethylfenoterol, (R,R′)-4′-aminofenoterol, (R,R′)-1-naphthylfenoterol and (R,R′)- and (R,S′)-4′-methoxy-1-naphthylfenoterol, were synthesized as previously described (Jozwiak et al, J Med Chem 50:2903-2915, 2007; Jozwiak et al, Bioorg Med Chem 18:728-736, 2010; each of which is incorporated by reference in its entirety). [³H]-Thymidine (70-90 Ci/mmol) was purchased from PerkinElmer Life and Analytical Sciences (Waltham, Mass.). Eagle's Minimum Essential Medium (E-MEM), trypsin solution, phosphate-buffered saline (PBS), fetal bovine serum (FBS), 100× solutions of sodium pyruvate (100 mM), L-glutamine (200 mM), and penicillin/streptomycin (a mixture of 10,000 units/ml penicillin and 10,000 μg/ml streptomycin) were obtained from Quality Biological (Gaithersburg, Md.). WIN 55,212-2, AM251, and AM630 were purchased from Cayman Chemical (Ann Arbor, Mich.). ICI 118,551 hydrochloride and (R)-isoproterenol were obtained from Sigma-Aldrich (St. Louis, Mo.). Phenylmethylsulfonyl fluoride (PMSF), benzamidine, leupeptin, pepstatin A, MgCl₂, EDTA, Trizma-Hydrochloride (Tris-HCl), (±)-propranolol and minimal essential medium (MEM) were obtained from Sigma Aldrich (St. Louis, Mo.). Egg phosphatidylcholine lipids (PC) were obtained from Avanti Polar Lipids (Alabaster, Ala.). (±)-fenoterol was purchased from Sigma-Aldrich and [³H]-(±)-fenoterol was acquired from Amersham Biosciences (Boston, Mass.). The organic solvents n-hexane, 2-propanol and triethylamine were obtained as ultra pure HPLC grade solvents from Carlo Erba (Milan, Italy). Fetal bovine serum and penicillin-streptomycin were purchased from Life Technologies (Gaithersburg, Md.), and [¹²⁵I]-(i)-iodocyanopindolol (ICYP) was purchased from NEN Life Science Products, Inc. (Boston, Mass.).

### Cell Culture

The human pancreatic cancer cell line (PANC-1), glioblastoma cell line U-87MG (U87MG), and breast cancer cell lines MDA-MB-231 and MCF-7 were purchased from ATCC (Manassas, Va.). Upon receipt of the cell lines, cells were expanded for a few passages to enable the generation of new frozen stocks. Cells were resuscitated as needed and used for fewer than 6 months after resuscitation (no more than 10 passages). ATCC performs thorough cell line authentication utilizing Short Tandem Repeat (STR) profiling. PANC-1 and U87MG cells were maintained in DMEM with L-glutamine supplemented with 10% FBS (Hyclone, Logan, Utah) and 1% penicillin/streptomycin. MDA-MB-231 cells were maintained in RPMI-1640 supplemented with 10% FBS and 1% penicillin/streptomycin and MCF-7 cells were maintained in EMEM with L-glutamine supplemented with 10% FBS and 0.01 mg/ml human recombinant insulin. Cells were maintained in a controlled environment (37° C. under humidified 5% CO₂ in air), and the medium was replaced every 2-3 days. Prior to experiments, cells were seeded on 100×20 mm tissue culture plates and grown to >70% confluency unless stated otherwise.

### Methods

Cell Treatment and Extraction—

The cells were seeded on 100×20 mm tissue culture plates and maintained at 37° C. under humidified 5% CO₂ in air until they reached >70% confluence. The original media was replaced with media containing 0.5 μM or 1 μM MNF and the plates were incubated for an additional 1 hour, unless otherwise indicated. After completion of incubations, the media was aspirated and the cells were washed twice with 10 ml of phosphate buffer saline (pH 7.4) (PBS). The cells were then collected in 10 ml PBS by scrapping and sedimented by centrifugation (1000 rpm, 5 min), and the supernatant discarded. The cell pellets were stored at −80° C. until analyzed.

The cell pellet was re-suspended in 1 ml of 80% methanol (cryogenically cold) and incubated at −80° C. for 15 minutes. This suspension was then centrifuged at 13,000×g for 10 minutes at 4° C. The supernatant was collected in an eppendorf tube and the pellet was suspended in 0.25 ml of water. The suspension was vortexed mixed for 1 minute and centrifuged at 13,000×g for 10 minutes at 4° C. The supernatant was collected and mixed with the supernatant collected in the previously step and stream dried under nitrogen. The sample was stored at −80° C. until analyzed.

Sample Preparation and NMR Analysis—Non-Targeted Metabolomics.

The cell pellet was re-suspended in 1 ml of 80% methanol (cryogenically cold) and incubated at −80° C. for 15 minutes. This suspension was then centrifuged at 13,000×g for 10 minutes at 4° C. The supernatant was collected in an eppendorf tube and the pellet was suspended in 0.25 ml of water. The suspension was vortexed mixed for 1 minute and centrifuged at 13,000×g for 10 minutes at 4° C. The supernatant was collected and mixed with the supernatant collected in the previously step and stream dried under nitrogen. The sample was stored at −80° C. until analyzed. The samples were prepared for NMR experimentation by dissolving in 0.6 ml of 50 mM phosphate buffer (pH 7.2) in 99.8% D₂O with 50 μM 3-(tetramethysilane)propionic acid-2,2,3,3-d4 (TMSP). NMR spectra were recorded on a Bruker Avance III-HD 700 MHz spectrometer equipped with a QCI-P cryoprobe and a SampleJet automated sample changer. 1D ¹H spectra were collected using excitation sculpting to remove the solvent signal. A total of 16 k data points with a spectral width of 5482.5 Hz, 8 dummy scans, and 256 scans were used to obtain each spectrum. The data was processed completely in MVAPACK.

[³H]-Thymidine incorporation studies. Cells were seeded in 24-well plates at 2.5×10⁴ cells per well and grown for 24 hours at 37° C. The effect of MNF (0-10 μM) on [³H]-thymidine incorporation into PANC-1, MDA-MB-231, and MCF-7 cells was investigated following procedures previously described in Paul, et al., Cannabinoid receptor activation correlates with the proapoptotic action of the β2-adrenergic agonist (R,R′)-4-methoxy-1-naphthylfenoterol in HepG2 hepatocarcinoma cells, J. Pharmacol. Exp. Ther., vol. 343, pgs 157-166 (2012); and Paul, et al., (R,R′)-4′-methoxy-1-naphthylfenoteroltargets GPR55-mediated ligand internalization and impairs cancer cellmotility, Biochem. Pharmacol., vol. 87, pgs 547-561 (2014). The effect of MNF on doxorubicin cytotoxicity in PANC-1 cells was measured using [³H]-thymidine incorporation. In these studies, the cells were incubated with either fresh medium containing vehicle (0.01% DMSO) or MNF (1 μM) for 24 hours. The spent media was removed and replaced with fresh media supplemented with doxorubicin (0-10 μM) and the cells incubated for an additional 24 hours. [³H]-Thymidine (1 μCi per well) incorporation into DNA was then performed for 16 hours. Cells were washed with PBS, lysed in 600 μt of 0.1 N NaOH, and the radioactivity was measured by liquid scintillation counting. Three independent experiments were conducted on separate days. Similar experiments were conducted to investigate the effect of MNF on the cytotoxicity of gemcitabine (0-10 μM) in PANC-1 cells. The effects of MNF (1 μM) on the cytotoxicity of doxorubicinin MDA-MB-231 cells and of MNF (0.01 μM) on the cytotoxicity of doxorubicin in U87MG cells were investigated using the protocol described above.

Western Blot Studies—Cytosolic Proteins.

In the first series of experiments, PANC-1 cells were incubated with media containing vehicle (0.01% DMSO) or MNF (10, 100, 500 and 1000 nM) for 24 hours. The medium was removed, and cells were collected and processed for immunoblot analysis. In a second series of experiments, PANC-1 cells were pretreated with vehicle, 1 μM MNF or 1 μM CID (GPR55 antagonist) for 30 minutes followed by the addition of 1 μM AM251 (GPR55 agonist) for 24 hours. Cell lysates were processed for immunoblot analysis, focusing on the expression of selected cancer cell biomarkers. In the third series of experiments, serum-starved PANC-1 cells were pretreated with vehicle (0.01% DMSO), 1 μM MNF, or 1 μM CID for 10 minutes followed by a 20 minute incubation with and without 1 AM251. Cell lysates were processed for immunoblot analysis and the levels of phospho-active (Ser473) and total forms of AKT were determined. All experiments were repeated three times on three separate days, unless stated otherwise.

Western Blot Studies—Constitutive and Inducible Nuclear Translocation of Select Cancer Biomarkers.

PANC-1 cells were serum starved for 3 hours prior to treatment with vehicle, 1 μM MNF, or 1 μM CID for 10 minutes followed by a 20 minute incubation with and without 1 μM AM251. Nuclear protein extraction was carried out using a NE-PER Kit followed by the determination of the levels of HIF-1α and phospho-active forms of β-catenin (Tyr333) and PKM2 (Ser37) by immunoblotting. The same experiment was repeated three times on three separate days.

Western Blot Analysis.

Cells were lysed in radioimmuno precipitation buffer containing EGTA and EDTA (Boston BioProducts, Ashland, Mass.) supplemented with protease inhibitor cocktail (Sigma-Aldrich). Protein concentration in clarified lysates was determined using the bicinchoninic acid reagent (Thermo Fisher Scientific). Proteins (20 μg/well) were separated on 4-12% precast gels (Invitrogen, Carlsbad, Calif.) using SDS-polyacrylamide gel electrophoresis under reducing conditions and then electrophoretically transferred onto polyvinylidenefluoride membrane (Invitrogen). Western blots were performed according to standard methods, which involved a blocking step in Tris-buffered saline/0.1% Tween-20 (TBS-T) supplemented with 5% non-fat milk and incubation with primary antibodies of interest. All membrane-bound primary antibodies were detected with horseradish peroxidase-conjugated secondary antibodies (Santa Cruz Biotechnology, Dallas, Tex.) and visualized by enhanced chemiluminescence (ECL Plus, GE Healthcare, Piscataway, N.J.). Quantification of the protein bands was performed by volume densitometry using ImageJ software (National Institutes of Health, Bethesda, Md.) and normalization to β-actin or Lamin A/C. The primary antibodies used in this study were raised against EGFR (sc-03), BCRP (sc-58222), MRP1 (sc-7773), MRP5 (sc-5780), cyclinD1 (sc-8396), β-catenin (sc-7199), and lamin A/C (sc-20681)(Santa Cruz Biotechnology); Pgp (ab129450), PKM2 (ab38237), HIF-1 α (ab51608), and β-actin (ab6276) (Abcam, Cambridge, Mass.); phospho-Ser473 AKT (4060) and AKT (4685) (Cell Signaling Technology, Beverly, Mass.); phospho-Tyr333 β-catenin (#11574) andphospho-Ser37 PKM2 (#11456) antibodies were purchased from Signalway Antibody, Co. (College Park, Md.). The antibodies were used at the dilution recommended by the manufacturers.

Measurement of nuclear phospho-PKM2. PANC-1 cells were seeded on Lab-Tek II CC2 chamber slides (Thermo Scientific Nunc, Rochester, N.Y.) and grown until they reached >70% confluence. The medium was removed and cells were incubated with serum-free medium for 3 hours, after which vehicle, 1 μM MNF or 1 μM CID was added for 10 minutes. The GPR55 agonist AM251 (1 μM) was added for 20 minutes where indicated. Cells were washed twice with PBS, fixed with 4% (v/v) paraformaldehyde (Sigma-Aldrich) in PBS for 20 minutes at room temperature, followed by 3×PBS washes and permeabilization with 0.1% Triton X-100 (Sigma-Aldrich) for 15 minutes. Next, the cells were incubated with 10% goat serum for 1 hour, followed by overnight incubation with rabbit polyclonal anti-phospho-PKM2 antibody (Ser37, 1:200) at 4° C. After 3×PBS washes, Alexa Fluor 488-conjugated goat anti-rabbit antibody (Invitrogen; 1:1000) was added to the slides for 2 hours at room temperature. The slides were washed with PBS and mounted in Prolong Gold antifade mounting medium containing DAPI (4′,6-diamidino-2-phenylindole) (Invitrogen) for nuclear counterstaining. The slides were cured for 24 hours at room temperature in the dark, after which images were acquired with a Zeiss LSM 710 confocal microscope using Carl Zeiss LSM software (Thorn-wood, NY). ImageJ software was used to quantify the changes in the nuclear pool of phospho-PKM2 levels.

Intracellular Uptake of Doxorubicin.

PANC-1 and MDA-MB-231 cells were seeded on Lab-Tek II CC2 chamber slides and grown until they reached >70% confluence. The original media was removed and cells were incubated in fresh media supplemented with vehicle (0.01% DMSO) or 1 μM MNF for 24 hours, after which doxorubicin (0.125 μM) was added for an additional 2 hours. The cells were washed with PBS and the images were acquired with a Zeiss axiovert 200 microscope using standard FITC filter (#09 filter set) and Carl Zeiss LSM software. ImageJ software was used to quantify the changes in the nuclear accumulation of doxorubicin defined as ring-shaped regions of interest (ROI).

Statistical Analysis.

Sigmoidal dose-response curves (IC₅₀ curves) were determined using the ‘nonlinear regression (curve fit)’ model contained within the Prism 4 software package (GraphPad Software, Inc., La Jolla, Calif.) running on a personal computer. For immunoblot analyses, statistical comparisons between treated and control groups were performed using unpaired Student's t-tests. Data are expressed as relative fold change ±standard deviation. P values ≤0.05 were considered significant.

### Example 1

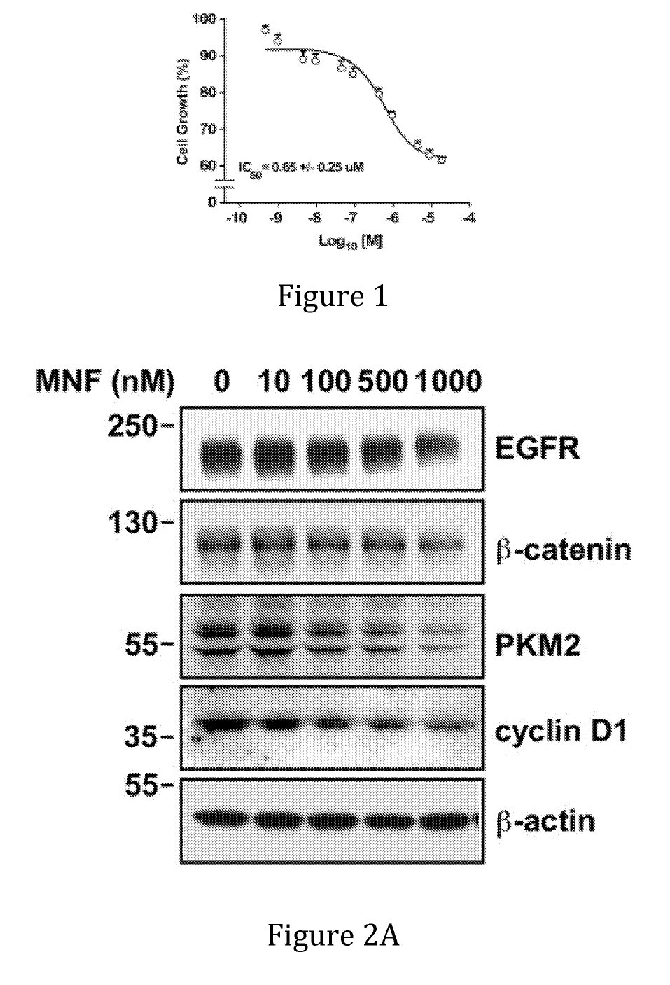

MNF was shown to inhibit proliferation and attenuate the expression of cancer biomarkers in PANC-1 cells. The effect of MNF on the proliferation of PANC-1 cells was assessed using a [³H]-thymidine incorporation assay. MNF produced a significant dose-dependent inhibition in cell growth (maximum of 42% reduction, p<0.001) with an IC₅₀ of 0.65±0.25 μM. (See, FIG. 1).

### Example 2

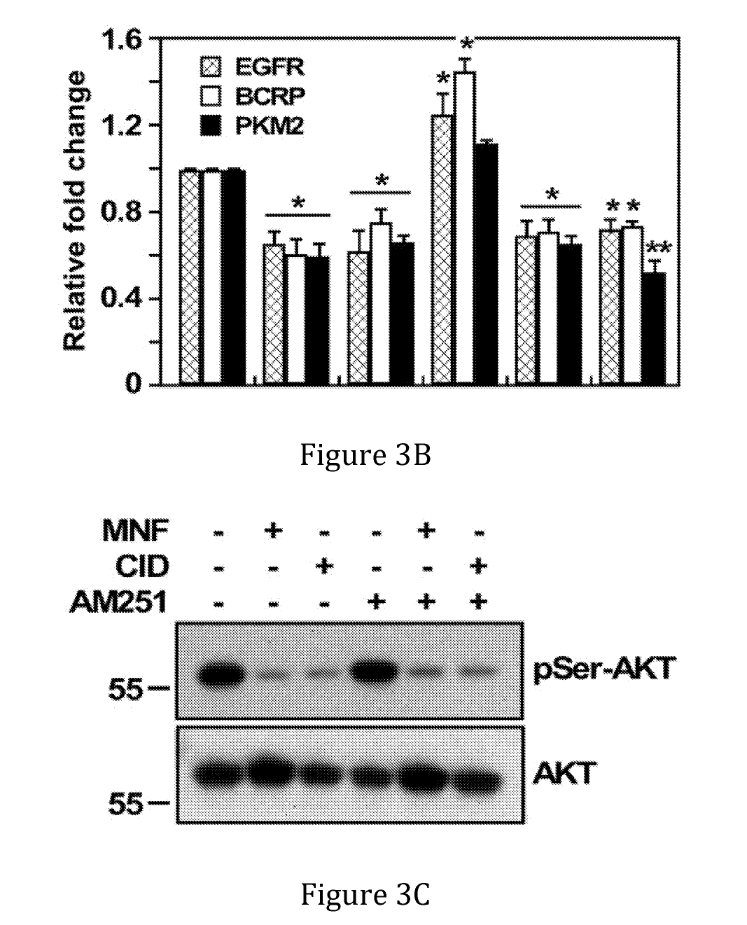

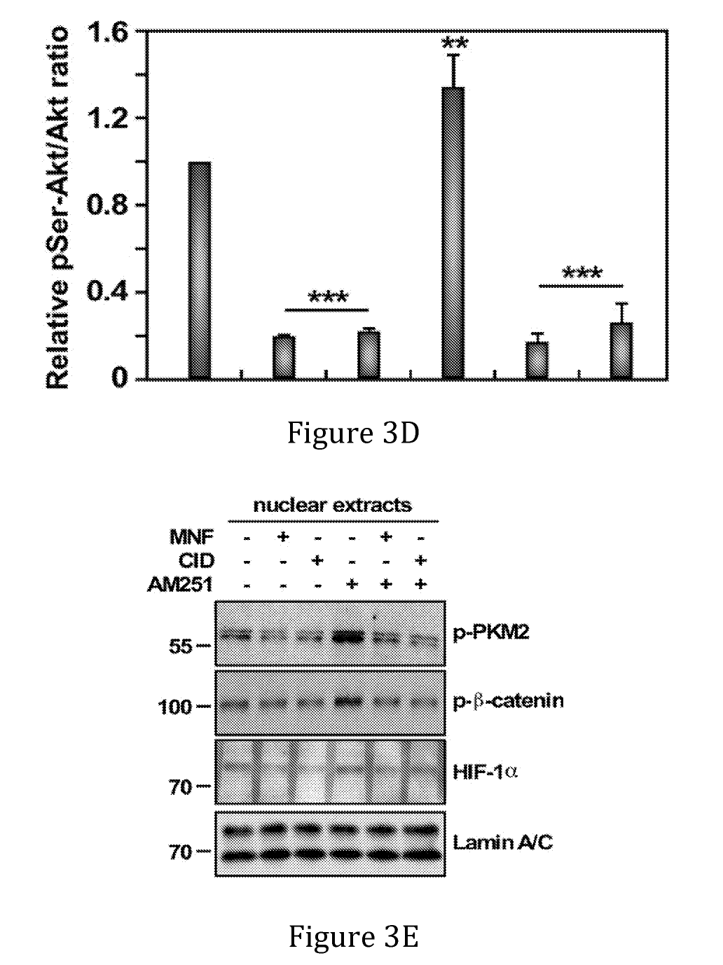

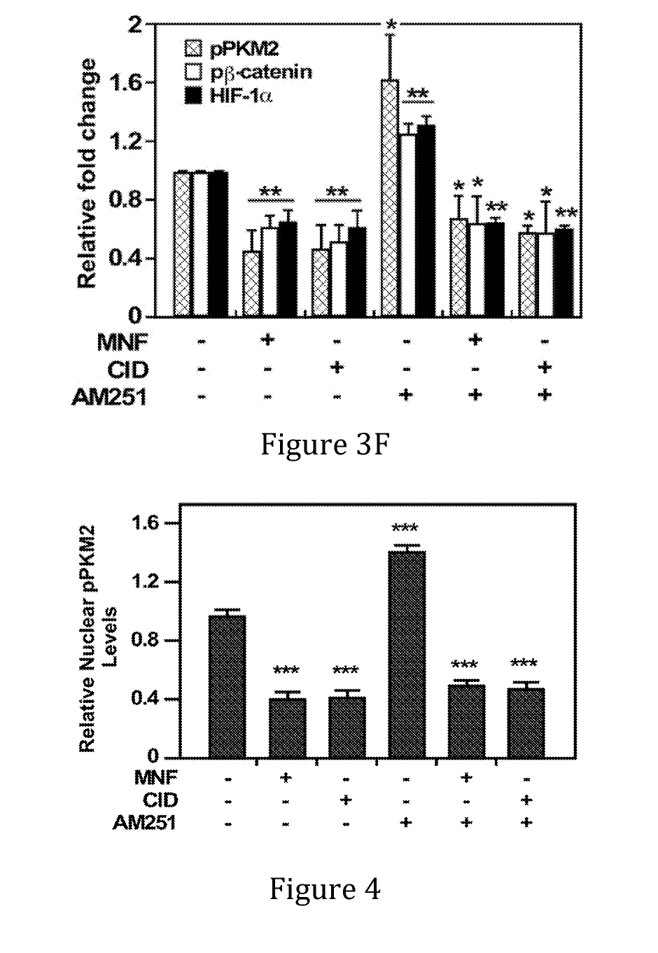

MNF is shown to exert antitumorigenic signaling through inhibition of GPR55. To confirm that the inhibition of GPR55 activity was involved in the antitumorigenic effects of MNF, PANC-1 cells were pretreated with CID, a characterized pharmacological inhibitor of GPR55, and then incubated with AM251, a potent GPR55 agonist. In a pattern similar to the one produced by MNF, cell treatment with CID significantly reduced the basal levels of EGFR, BCRP, and PKM2 proteins and blocked the induction of these cancer biomarkers by AM251 (FIGS. 3A and B). Upon exposure to MNF and CID, the activating phosphorylation of AKT was significantly decreased both basally and after cell treatment with AM251. (See, FIGS. 3C and D). Moreover, constitutive and AM251-induced nuclear accumulation of HIF-1α and phos-phoactive forms of PKM2 (Ser37) and β-catenin (Tyr333) was significantly abrogated in PANC-1 cells after 10 minutes of MNF and CID treatment. (See, FIGS. 3E and F). Without wishing to be bound by any theory, it is believed that the ability of MNF to down-regulate the expression of cancer biomarkers in GPR55-expressing cancer cells may stem from inhibition of the transcriptional activity of PKM2, β-catenin and/or HIF-1α. Immunofluorescence confocal images (not shown) revealed nuclear pool of phospho-active PKM2 (p-PKM2) after cell pretreatment with MNF (1 μM) or CID (1 μM) for 10 minutes followed by a 20 minute treatment without (control) and with AM251 (1 μM) and showed the accumulation of nuclear p-PKM2 or its lack thereof in response to various treatments. Thus, immunofluorescence confocal microscopy experiments established that acute treatment with either MNF or CID markedly reduced the nuclear pool of phosphoactive form of PKM2 both in basal conditions and following cell stimulation with the GPR55 agonist, AM251. Quantitative analysis of the immunofluorescence data is presented in FIG. 4.

### Example 3

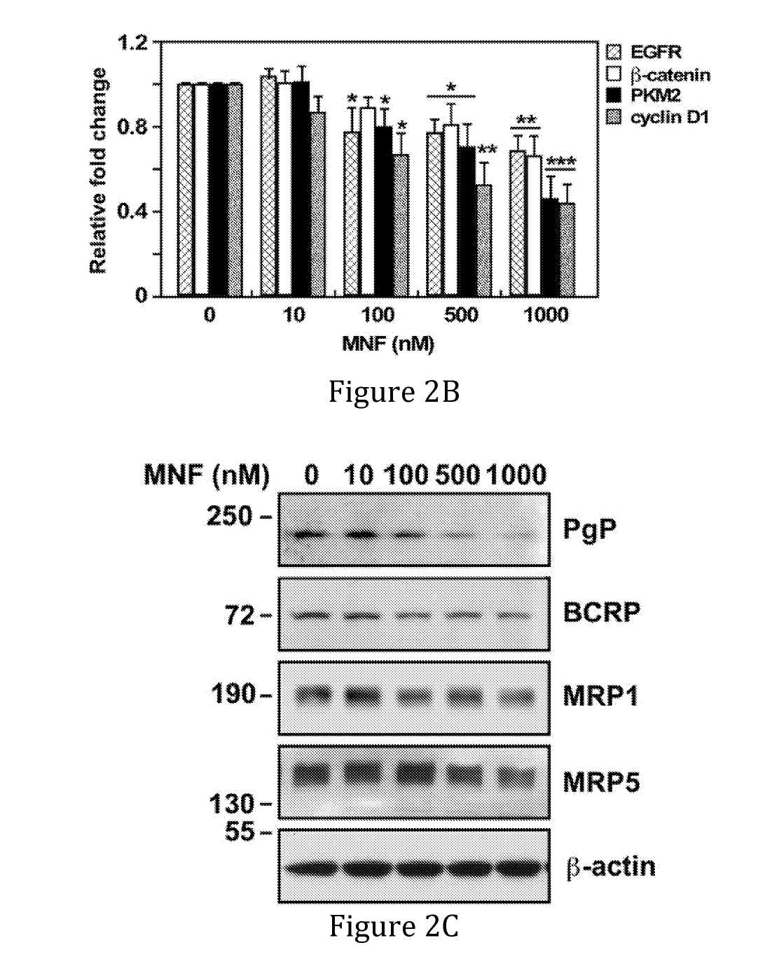

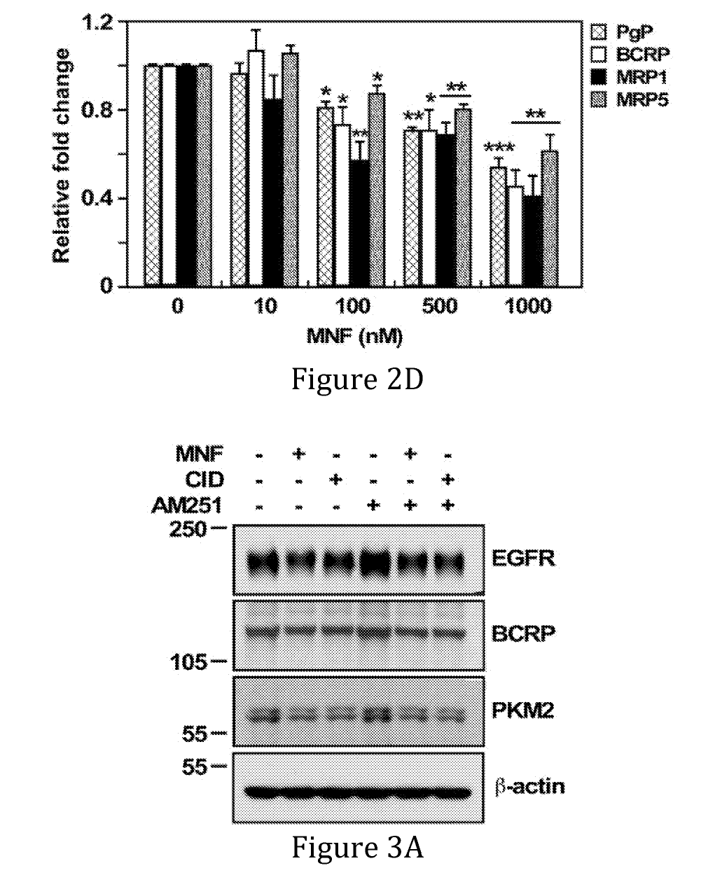

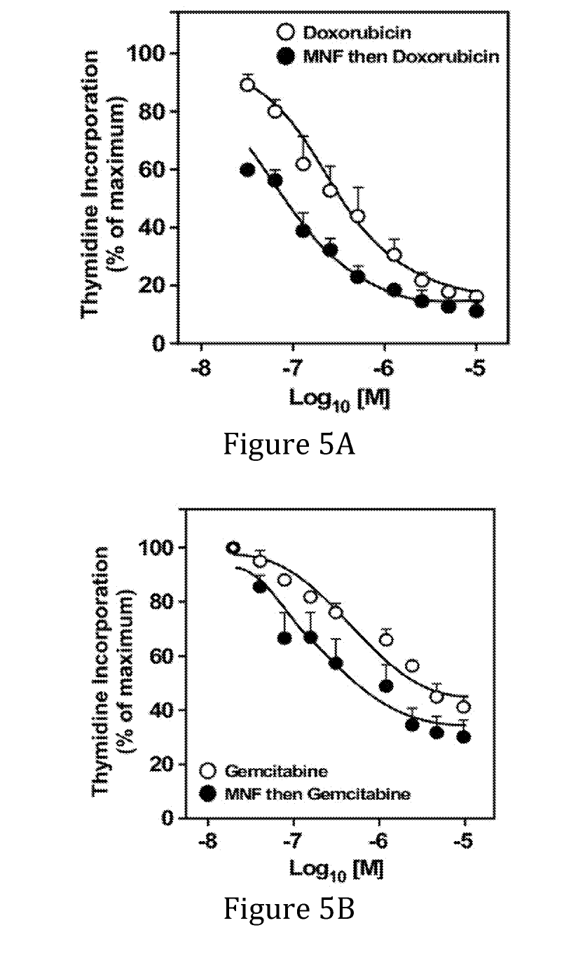

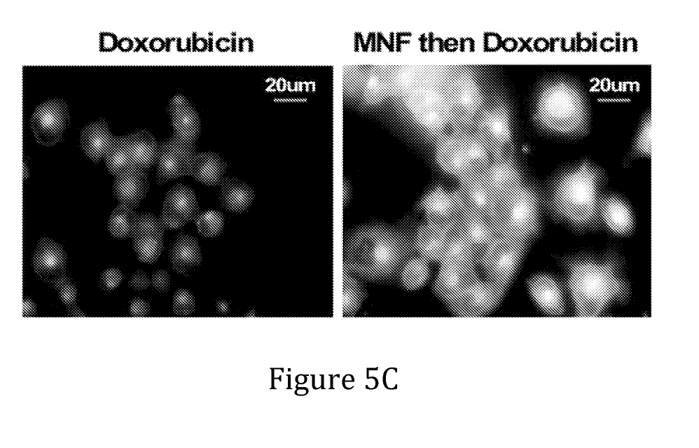

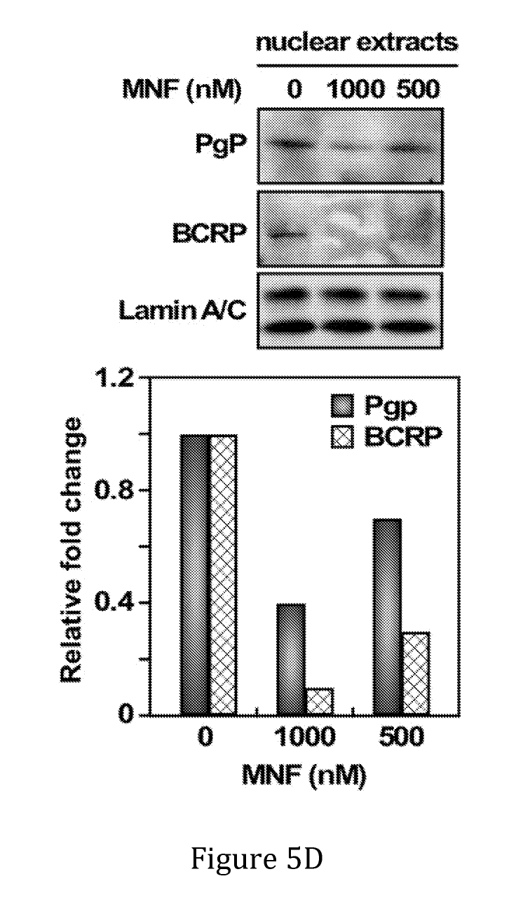

MNF was shown to increase the cytotoxicity of chemotherapeutic drugs inPANC-1 cells by suppressing expression of multidrug resistance proteins. To determine if the MNF-associated reduction of the endogenous expression of Pgp, BCRP, MPR1 and MRP5 in PANC-1 cells (see, FIGS. 2C and D) had functional consequences, the effect of MNF pretreatment on the anti-proliferative response of doxorubicin was assessed using a [³H]-thymidine incorporation assay. Doxorubicin is a prototypical substrate of Pgp and MDR1-5 transporters and a key probe of the MDR phenotype. In order to control for competitive inhibition of MDR transport by MNF, the PANC-1 cells were incubated with MNF for 24 hours and the spent media was then replaced with fresh culture medium containing only doxorubicin. The data demonstrate that MNF pretreatment significantly enhanced doxorubicin cytotoxicity resulting in ˜4-fold decrease in the calculated IC₅₀ value from 0.193±0.004 μM to 0.051±0.002 μM. (See, FIG. 5A, p<0.001). Resistance to the cytotoxic effects of gemcitabine, a frontline chemotherapeutic agent in pancreatic cancer treatment, has been associated with MRP5-mediated export of the drug. In view of the ability of MNF to reduce MRP5 expression in PANC-1 cells (see, FIGS. 2C and D), the cytotoxic response of gemcitabine +/− MNF was investigated using the same protocol utilized with doxorubicin. The results indicate that MNF pretreatment produced a ˜3-fold decrease in the calculated IC₅₀ value of gemcitabine from 0.487±0.003 μM to 0.153±0.003 μM. (See, FIG. 5B, p<0.001). In view of the intrinsic fluorescence of doxorubicin, its cellular distribution was evaluated using fluorescence imaging microscopy. The results indicated an enhancement in the uptake and nuclear retention of doxorubicin in MNF-treated PANC-1 cells compared to control cells. (See, FIG. 5C). Immunoblots of nuclear extracts revealed a marked reduction in the nuclear pool of Pgp and BCRP, two MDR-related proteins, following a 24 hour treatment of PANC-1 cells with MNF. (See, FIG. 5D).

### Example 4

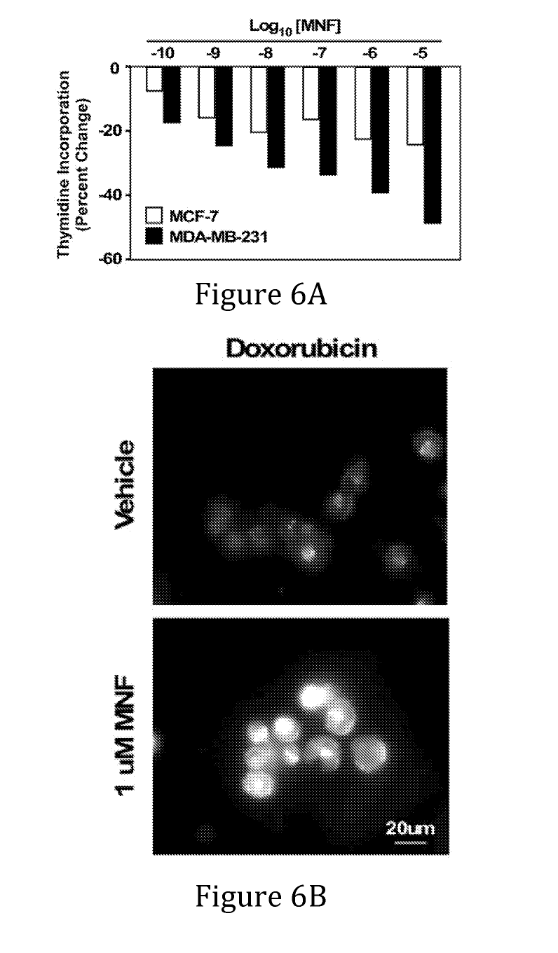

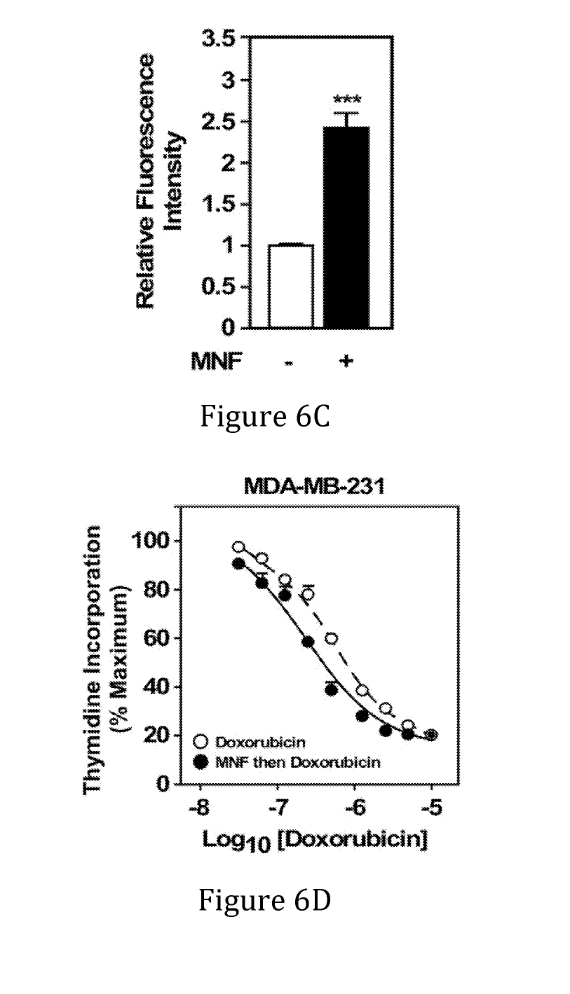

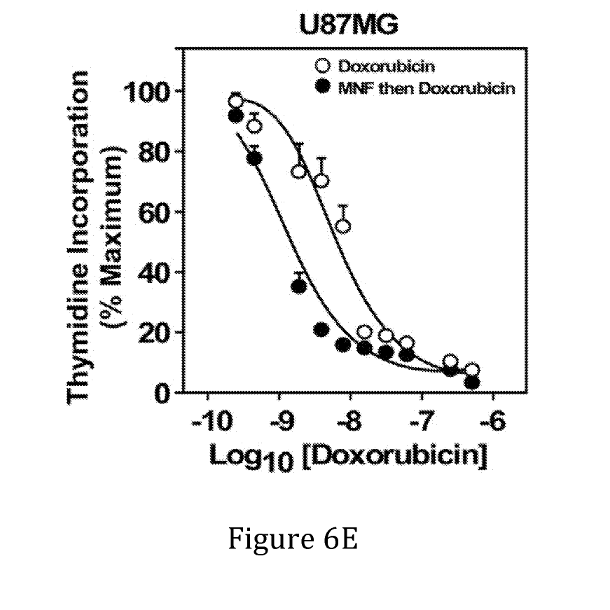

Pretreatment with MNF was shown to increase doxorubicin cytotoxicity in MDA-MB-231 breast cancer cells and U87MG glioblastoma cells. The effect of MNF pretreatment on doxorubicin cytotoxicity was examined in two additional human tumor-derived cell lines using MDA-MB-231 breast cancer cells and U87MG glioblastoma cells. The MDA-MB-231 cell line demonstrates a high expression of functional GPR55, and human breast cancer cells MCF-7 express low levels of GPR55. The effect of MNF on the proliferation of MDA-MB-231 and MCF-7 cells was assessed using a [³H]-thymidine incorporation assay. MNF incubation with MDA-MB-231 cells produced a 50% dose-dependent inhibition in cell growth with an IC₅₀ of 0.106±0.003 μM, but it had no significant effect on the growth of MCF-7 cells. (See, FIG. 6A). As observed with PANC-1 cells, MNF pre-treatment resulted in an enhanced uptake and nuclear retention of doxorubicin in MDA-MB-231 cells compared to control cells. (See, FIGS. 6, B and C). Consistent with this observation, incubation of MDA-MB-231 cells with MNF for 24 hours followed by replacement with fresh culture medium containing only doxorubicin produced a significant enhancement of doxorubicin cytotoxicity as the calculated IC₅₀ value decreased from 0.570±0.014 μM to 0.255±0.013 μM. (See, FIG. 6D, p<0.05). In this study, incubation of U87MG cells with MNF for 24 hours followed by replacement with fresh culture medium containing only doxorubicin, produced a significant enhancement of doxorubicin cytotoxicity as the calculated IC₅₀ value decreased from 0.0060±0.0008 μM to 0.0011±0.0005 μM. (See, FIG. 6E, p<0.001).

Methods in accordance with certain embodiments of the present disclosure advantageously employ GPR55 antagonism to decrease the expression of Pgp, BCRP, MRP1 and MRP5 in cancer cells, in embodiments in pancreatic cancer cells. Moreover, treatment of cancer cells (in embodiments in pancreatic cancer cells) with the GPR55 agonist AM251 produces a significant increase in BCRP expression, which was blocked in accordance with certain embodiments of the methods described herein by the two GPR55 antagonists tested, MNF and CID (see, FIG. 4).

In another aspect, contacting cancer cells (in embodiments in pancreatic cancer cells) with MNF and/or CID in accordance with certain embodiments of the methods described herein reduces nuclear PKM2 levels both under basal conditions and upon GPR55 stimulation.

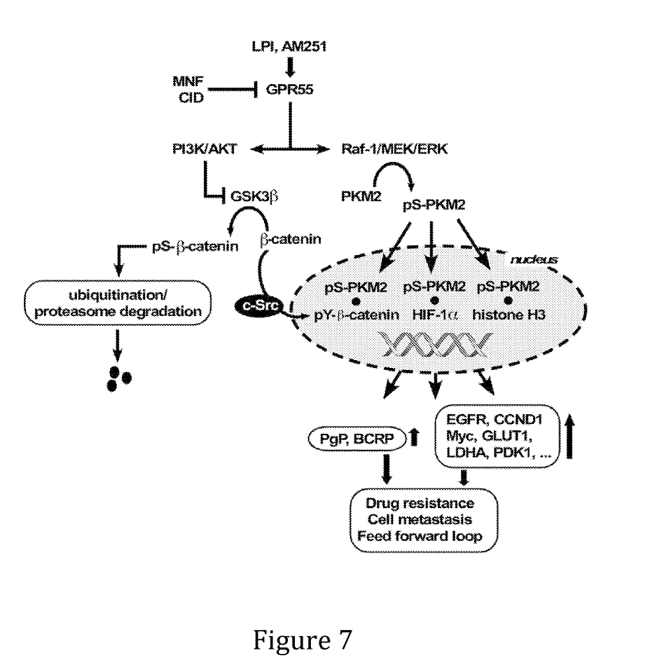

While not wishing to be bound by any theory, this effect is consistent with the GPR55/MEK/ERK pathway resulting in the ERK-mediated phosphorylation of PKM2 at Ser 37, which is necessary for PKM2 nuclear translocation. It follows that the impaired formation of multiprotein complexes involving nuclear PKM2 with tyrosine-phosphorylated β-catenin and/or HIF-1α combined with lower phosphorylation of histone H3 may be responsible for the down regulation in the expression of tumor biomarkers, including MDR proteins, EGFR, and cyclin D1 as well as PKM2. Although cyclin D1 is a target gene of β-catenin (see, FIG. 1A), the effect of MNF on cyclin D1 was more sensitive than β-catenin. Thus, the results presented herein provide mechanistic insights into the suppression of EGFR, BCRP, PKM2, and other cancer biomarkers in response to pharmacological inhibition of GPR55 with MNF and CID (FIG. 7).

The effect of administering GPR55 antagonists such as MNF in accordance with certain embodiments of the methods described herein has a negative impact on the MEK/ERK pathway-dependent phosphorylation and nuclear translocation of PKM2, thereby reducing pro-oncogenic gene expression. Administering GPR55 antagonists such as MNF in accordance with certain embodiments of the methods described herein may also activate a feed-forward loop in which GPR55 activation leads to increased EGFR and PKM2 expression, thus potentiating the EGFR-MEK/ERK-PKM2 effects on proliferation and MDR (FIG. 7).

Upon agonist engagement of GPR55 with L-α-lysophosphatidylinositol (LPI) or AM251, there is increase in PI3K/AKT and c-Raf/MEK/ERK signaling pathways, which are responsible for the activation and nuclear translocation of β-catenin and PKM2, and formation of transcriptionally competent complexes on the promoters of pro-oncogenic target genes. The inhibition of GSK3β by AKT stabilizes β-catenin by preventing its phosphorylation-coupled proteolytic degradation. Pharmacological inhibition of GPR55 with GPR55 antagonists (such as MNF, alone or in combination with CID) promotes the dephosphorylation of both signaling pathways with subsequent impairment in PKM2, β-catenin and HIF-1α-dependent regulation of PanCa cell chemoresistance and behavior.

Administering GPR55 antagonists such as MNF in accordance with certain embodiments of the methods described herein also activates the PI3K/AKT signaling pathway, leading to the attenuated expression and nuclear accumulation of β-catenin and HIF-1α. (See, FIG. 7). The effectiveness of chemotherapy in a broad spectrum of tumors is limited by a multitude of factors including transporter-mediated MDR. For example, incubation of human-derived pancreatic cancer tumor cell lines with chemotherapeutic agents, such as gemcitabine, 5-fluorouracil, or cisplatin, results in an elevation of MDR protein expression in surviving cells and MDR-based resistance to chemotherapeutic agents in cancer therapy, such as, for example, in breast cancer therapy. Administering GPR55 antagonists such as MNF, in accordance with certain embodiments of the methods described herein provides a clinical treatment of MDR. Administering GPR55 antagonists such as MNF, alone or in combination with other GPR55 antagonists such as CID, in accordance with certain embodiments of the methods described herein reduces the total and nuclear expression of MDR proteins and reduces nuclear expression of Pgp and MRP1 in cancer cells (in embodiments in pancreatic cancer cells), resulting in increased nuclear accumulation of chemotherapeutic agents (such as doxorubicin) and increased cytotoxicity of chemotherapeutic agents (such as doxorubicin or gemcitabine). An increase in cellular and nuclear accumulation of chemotherapeutic agents in breast cancer cells and/or increase in chemotherapeutic agents' cytotoxicity in cancer cells can be achieved by administering GPR55 antagonists such as MNF, alone or in combination with other GPR55 antagonists, in accordance with certain embodiments of the methods described herein.

The methods described herein provide a bimodal model activity of GPR55 antagonists (such as MNF and others) in cancer cells (in embodiments in pancreatic cancer cells) (see, FIG. 7 regarding pancreatic cancer cells) and other GPR55-expressing tumors. Administering GPR55 antagonists such as MNF, alone or in combination with other GPR55 antagonists, in accordance with certain embodiments of the methods described herein attenuates both the constitutive and ligand-inducible activity of the MEK/ERK and PI3K/AKT pathways, which results in reduced phosphorylation and nuclear translocation of PKM2 and concomitant decrease in HIF-1 α and β-catenin levels. This, in turn, significantly downregulates MDR protein expression and disrupts feed-forward loops responsible for chemoresistance and survival in tumor cells.

While several embodiments have been described, it is not intended that the disclosure be limited thereto, as it is intended that the disclosure be as broad in scope as the art will allow and that the specification be read likewise. Other elements, steps, methods and techniques that are insubstantially different from those described above and/or in the appended claims are also intended to be within the scope of the disclosure. Therefore, the above description should not be construed as limiting, but merely as exemplifications of presently disclosed embodiments.

Persons skilled in the art will understand that the materials and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure. As well, one skilled in the art will appreciate further features and advantages of the present disclosure based on the above-described embodiments. Accordingly, the present disclosure is not to be limited by what has been particularly shown and described, except as indicated by the appended claims.

What is claimed is:

1. A method comprising:
administering a therapeutically effective amount of a pharmaceutical composition containing a pharmaceutically acceptable carrier and a compound that is a GPR55 antagonist to a subject having a tumor including cancer cells that have developed a resistance to one or more chemotherapeutic agents.

2. The method of claim 1 wherein the pharmaceutical composition is administered to a subject having a tumor including either pancreatic cancer cells or breast cancer cells that have developed a resistance to one or more chemotherapeutic agents.

3. The method of claim 1 wherein the pharmaceutical composition is administered to a subject having a tumor including cancer cells that have developed a resistance to at least one of doxorubicin or gemcitabine.

4. The method of claim 1 wherein the method further comprises administering a chemotherapeutic agent either before, after, or during the administration of the pharmaceutical composition.

5. The method of claim 1 wherein the pharmaceutical composition administered contains a fenoterol analogue.

6. The method of claim 1 wherein the pharmaceutical composition administered contains one or more compounds selected from the group consisting of (R,R′)-4′-methoxy-1-naphthylfenoterol (“MNF”), (R,S′)-4′-methoxy-1-naphthylfenoterol, (R,R′)-ethylMNF, (R,R′)-napthylfenoterol, (R,S′)-napthylfenoterol, (R,R′)-ethyl-naphthylfenoterol, (R,R′)-4′-amino-1-naphthylfenoterol, (R,R′)-4′-hydroxy-1-naphthylfenoterol, (R,R′)-4′-methoxy-ethylfenoterol, (R,R′)-4′-methoxyfenoterol, (R,R′)-ethylfenoterol, (R,R′)-fenoterol and their respective stereoisomers.

7. The method of claim 1 wherein the pharmaceutical composition administered contains a compound of the formula:

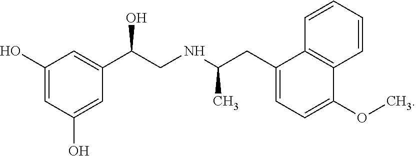 [SMILES: COc1ccc(C[C@@H](C)NC[C@H](O)c2cc(O)cc(O)c2)c2ccccc12]

8. The method of claim 1 wherein the pharmaceutical composition attenuates both the constitutive and ligand-inducible activity of the MEK/ERK and PI3K/AKT pathways.

9. The method of claim 1 wherein the pharmaceutical composition downregulates MDR protein expression.

10. The method of claim 1 wherein the pharmaceutical composition decreases HIF-1α and β-catenin levels in cancer cells of a tumor.

11. The method of claim 1 wherein the pharmaceutical composition results in increased nuclear accumulation of one or more chemotherapeutic agents in cancer cells of a tumor.

12. The method of claim 1 the pharmaceutical composition results in increased cytotoxicity of one or more chemotherapeutic agents with respect to the cancer cells of a tumor.

13. A method comprising:
identifying a subject having a tumor including cancer cells that have developed a resistance to one or more chemotherapeutic agents; and
administering to the subject a therapeutically effective amount of a pharmaceutical composition containing a pharmaceutically acceptable carrier and a compound that is a GPR55 antagonist.

14. The method of claim 13 wherein the subject identified has a tumor including either pancreatic cancer cells or breast cancer cells that have developed a resistance to one or more chemotherapeutic agents.

15. The method of claim 13 wherein the pharmaceutical composition is administered to a subject having a tumor including cancer cells that have developed a resistance to at least one of doxorubicin or gemcitabine.

16. The method of claim 13 wherein the pharmaceutical composition is administered together with a chemotherapeutic agent.

17. The method of claim 13 wherein the pharmaceutical composition is administered together with the chemotherapeutic agent to which the cancer cells of the tumor have developed a resistance.

18. The method of claim 13 further comprising administering an anticancer drug selected from the group consisting of doxorubicin, daunorubicin, mitoxantrone, paclitaxel, docetaxel, vincristine, vinblastine, cisplatin, methotrexate, gemcitabine, 5-Fluorouracil, bortezomib, carfilzomib, erlotinib, ceritinib, sunitib, imatinib, irinotecan, topotecan, palbociclib, and vemurafenib, the anticancer drug being administered before, after, or during administration of the pharmaceutical composition.

19. The method of claim 13 wherein the pharmaceutical composition administered contains one or more compounds selected from the group consisting of (R,R′)-4′-methoxy-1-naphthylfenoterol (“MNF”), (R,S′)-4′-methoxy-1-naphthylfenoterol, (R,R′)-ethylMNF, (R,R′)-napthylfenoterol, (R,S′)-napthylfenoterol, (R,R′)-ethyl-naphthylfenoterol, (R,R′)-4′-amino-1-naphthylfenoterol, (R,R′)-4′-hydroxy-1-naphthylfenoterol, (R,R′)-4′-methoxy-ethylfenoterol, (R,R′)-4′-methoxyfenoterol, (R,R′)-ethylfenoterol, (R,R′)-fenoterol and their respective stereoisomers.

20. The method of claim 13 wherein the pharmaceutical composition administered contains a compound of the formula:

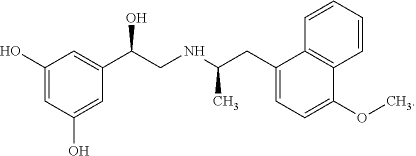 [SMILES: COc1ccc(C[C@@H](C)NC[C@H](O)c2cc(O)cc(O)c2)c2ccccc12]

21. The method of claim 13 wherein administering the pharmaceutical composition results in increased nuclear accumulation of one or more chemotherapeutic agents in the cancer cells.

22. The method of claim 13 wherein administering the pharmaceutical composition results in increased cytotoxicity of one or more chemotherapeutic agents with respect to the cancer cells.
